(19) United States
(12) Patent Application Publication
(10) Pub. No.: US 2022/0409772 A1
(43) Pub. Date: Dec. 29, 2022

(54) NANOCRYSTALLINE HYDROXYAPATITE/POLYURETHANE HYBRID POLYMERS AND SYNTHESIS THEREOF

(71) Applicant: Vanderbilt University, Nashville, TN (US)

(72) Inventors: Scott A. Guelcher, Nashville, TN (US); Sichang Lu, Nashville, TN (US); Madison A.P. McGough, Nashville, TN (US); Katarzyna J. Zienkiewicz, Nashville, TN (US)

(21) Appl. No.: 17/715,829

(22) Filed: Apr. 7, 2022

Related U.S. Application Data
(63) Continuation of application No. 15/768,810, filed on Apr. 16, 2018, now abandoned.
(60) Provisional application No. 62/242,288, filed on Oct. 15, 2015.

(51) Int. Cl. A61L 27/48; A61L 27/54; A61L 27/56; A61L 27/58; A61L 27/46; C08G 18/77; C08G 18/48; A61L 27/10; A61L 27/18; A61L 27/20; A61L 27/26; C04B 26/16; C08G 18/10; C08G 18/38; C08G 18/80

(52) U.S. Cl. CPC A61L 27/48; A61L 27/54; A61L 27/56; A61L 27/58; A61L 27/46; C08G 18/771; C08G 18/4833; A61L 27/10; A61L 27/18; A61L 27/20; A61L 27/26; C04B 26/16; C08G 18/10; C08G 18/3868; C08G 18/8061; A61L 2300/404; A61L 2400/06; A01K 2207/20; A61L 2300/412; A61L 2300/604; A61L 2400/12; A61L 2430/02; C04B 2111/00836

(57) ABSTRACT

A hybrid composite and method for producing a polymer network are provided. The hybrid composite includes nanocrystalline hydroxyapatite (nHA) and polyurethane. The method for producing a polymer network includes reacting nanocrystalline hydroxyapatite (nHA) particles with lysine derived triisocyanate (LTI) to form a nHA/LTI hybrid prepolymer and reacting the prepolymer with a thioketal (TK) diol to form a nHA/poly(thioketal urethane) (PTKUR) hybrid polymer network.

### RELATED APPLICATIONS

This application is a continuation of U.S. application Ser. No. 15/768,810, filed Apr. 16, 2018, which is a § 371 National Stage Application of PCT/US2016/057425, filed Oct. 17, 2016, which claims the benefit of U.S. Provisional Application Ser. No. 62/242,288, filed Oct. 15, 2015, the entire disclosures of which are incorporated herein by this reference.

### GOVERNMENT SUPPORT

This invention was made with government support under Grant No. AR064304 and CA163499 awarded by the NIH. The government has certain rights in the invention.

### TECHNICAL FIELD

The presently-disclosed subject matter relates to hybrid polymers and synthesis thereof. More specifically, the presently-disclosed subject matter relates to nanocrystalline hydroxyapatite (nHA)/polyurethane (PUR) hybrid composites and synthesis thereof.

### BACKGROUND

Bone cements utilized in the clinical management of fractures at weight-bearing sites, such as intra-articular joints, are subjected to repetitive, dynamic physiological loading from daily activities. Treatment of these fractures requires extensive open reduction and internal fixation devices along with subchondral grafting to stabilize the fracture, which is associated with high rates of complications, such as non-union and loss of reduction. Thus, there is a compelling clinical need for bone cements that stabilize intra-articular fractures with less hardware by optimizing structural compatibility with bone.

One common bone cement includes poly(methyl methacrylate) (PMMA) bone cement, which is intended for use in arthoplastic procedures for the fixation of prosthetic implants to bone. PMMA provides structural compatibility with bone based upon established performance criteria including compressive strength of 70-90 MPa, compressive modulus of 2000-3000 MPa, and bending strength 80 MPa. However, PMMA is non-resorbable, and tesorbable cement materials that combine both mechanical and biological properties of bone are not currently available. Additionally, the specific mechanical properties required for these materials to optimize structural compatibility with bone have yet to be established.

In an attempt to design resorbable cements with mechanical properties comparable to or exceeding those of PMMA, recent studies have highlighted the enhanced remodeling and mechanical properties of biphasic bone cements compared to monophasic cements. Reinforcement of calcium phosphate cements with polymer or metal fibers can increase the toughness of the material by up to two orders of magnitude (bending strength 139 MPa). However, there are a limited number of preclinical studies evaluating remodeling of these materials in bony defects.

Another possibility may include inorganic-organic hybrid polymers, which exhibit enhanced mechanical properties. Inorganic-organic hybrid polymers incorporating inorganic nanoparticles bound to the organic component are microscopically phase-separated but macroscopically uniform, and consequently exhibit improved nanoparticle dispersion and increased mechanical properties compared to physically mixed composites. As an example, polyurethane-polyhedral oligomeric silsesquioxane (POSS) hybrid polymers showed enhanced mechanical properties and thermal stability compared to physically-mixed POSS composites due to increased POSS-polyurethane interactions. Although the incorporation of ceramic microparticles in organic polymers to form composites has been extensively investigated, structure-property relationships for nanocomposite materials are not well known.

Additionally, while nHA-collagen hybrids have been reported, hybrids with hydrophobic polymers have not been extensively investigated. The hydroxyl (P—OH) group on the surface of nHA is a reactive group that can be used to graft organic molecules, including polyisocyanates such as hexamethylene diisocyanate. However, the use of nHA prepolymers to synthesize injectable and settable hybrid polymers has not been previously reported. The effect of using such a prepolymer on the mechanical and biological properties of the resulting nHA-polyisocyanate/poly(ester urethane) cement has also not been investigated.

Furthermore, for polymer/ceramic composites in which the ceramic forms a dispersed particulated phase that provides an osteoconductive scaffold for new bone formation, the rates of new bone formation and polymer and ceramic resorption must be balanced to avoid resorption gaps and fibrous scar formation. For large defects, hydrolytically degradable polymers such as polyesters are limited by premature polymer degradation in the interior of the composite before the cells have infiltrated. Currently available composite BVFs comprise apatitic cements, polymers, and/or degradable metals, which have limited biological activity. nHA with grain size <100 nm enhances osteogenic differentiation, new bone formation, and osteoclast differentiation compared to amorphous or micron-scale crystalline hydroxyapatite. However, the effects of grafting reactive polymers to the nHA surface on its biological activity have not been extensively investigated.

Hence, there remains a need for a BVF that enhances bone cell activity, exhibits bone-like strength, and is hydrolytically stable but cell-degradable.

### SUMMARY

The presently-disclosed subject matter meets some or all of the above-identified needs, as will become evident to those of ordinary skill in the art after a study of information provided in this document.

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned, likewise, those features can be applied to other embodiments of the presently disclosed subject matter, whether listed in this Summary or not. To avoid excessive repetition, this Summary does not list or suggest all possible combinations of such features.

In some embodiments, the presently-disclosed subject matter includes a hybrid composite. In one embodiment, the hybrid composite includes nanocrystalline hydroxyapatite (nHA) and polyurethane. The polyurethane includes any suitable polyurethane, such as, but not limited to, poly(thioketal urethane) (PTKUR), poly(ester urethane), lysine-derived polyurethane, and/or any combination thereof. In another embodiment, the composite is resorbable, injectable, and/or settable. Additionally or alternatively, the composite is moldable. In one embodiment, the moldable composite includes at least one additive. In another embodiment, the additive is a granular particle, such as, but not limited to, a ceramic granule, a porogen, and/or a combination thereof. In a further embodiment, the ceramic granules are slowly degrading ceramic granules having a size of between 100 and 300 μm. Such ceramic granules may be arranged and disposed to facilitate osseointegration in a subject.

In certain embodiments, the composite includes between between 20 and 65 wt % nHA, including, but not limited to, at least 50 wt % nHA, at least 60 wt % nHA, or at least 65 wt % nHA. The composite may also include at least one anti-microbial and/or osteobiologic. In some embodiments, the composite is hydrolytically stable. In some embodiments, the composite is arranged and disposed to undergo cell-mediated oxidation of lysine and thioketal (TK) residues while nHA is resorbed by osteoclasts. Additionally, the composite according to one or more of the embodiments disclosed herein may form a bone void filler.

The presently-disclosed subject matter also includes A method for producing a polymer network including reacting nanocrystalline hydroxyapatite (nHA) particles with lysine derived triisocyanate (LTI) to form a nHA/LTI hybrid prepolymer and reacting the prepolymer with a thioketal (TK) diol to form a nHA/poly(thioketal urethane) (PTKUR) hybrid polymer network. In some embodiments, the nHA particles are less than 100 nm. Additionally or alternatively, in some embodiments, the nHA particles are reacted with the LTI at a NCO:OH ratio of between about 20:1 to about 3:1. In certain embodiments, the nHA particles have a specific surface of greater than 10 m² g⁻¹.

In some embodiments, the prepolymer in the method is 65 wt % nHA. In one embodiment, the polymer network is 55% nHA. In another embodiment, the TK diol is hydrolytically stable and oxidatively degradable. In a further embodiment, the TK diol includes thioketal bonds that are destabilized by hydroxyl radicals. The destabilization of the thioketal bonds may facilitate chain scission and/or breakdown to original monomers.

Further advantages of the presently-disclosed subject matter will become evident to those of ordinary skill in the art after a study of the description, Figures, and non-limiting Examples in this document.

### Definitions

While the following terms used herein are believed to be well understood by one of ordinary skill in the art, definitions are set forth to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a composition” includes a plurality of such compositions, and so forth.

Unless otherwise indicated, all numbers expressing quantities, properties, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The terms, “biodegradable”, “bioerodable”, or “resorbable” materials, as used herein, are intended to describe materials that degrade under physiological conditions to form a product that can be metabolized or excreted without damage to the subject. In certain embodiments, the product is metabolized or excreted without permanent damage to the subject. Unless otherwise stated herein, biodegradable materials may be hydrolytically degradable, may require cellular and/or enzymatic action to fully degrade, or both. Biodegradable materials also include materials that are broken down within cells. Degradation may occur by hydrolysis, oxidation, enzymatic processes, phagocytosis, or other processes. Some degradation may occur due to the present of reactive oxygen species.

The term “biocompatible” as used herein, is intended to describe materials that, upon administration in vivo, do not induce undesirable side effects. In some embodiments, the material does not induce irreversible, undesirable side effects. In certain embodiments, a material is biocompatible if it does not induce long term undesirable side effects. In certain embodiments, the risks and benefits of administering a material are weighed in order to determine whether a material is sufficiently biocompatible to be administered to a subject.

The term “composite” as used herein, is used to refer to a unified combination of two or more distinct materials. The composite may be homogeneous or heterogeneous. For example, a composite may be a combination of bone particles and a polymer; a combination of bone particles, polymers and antibiotics; or a combination of two different polymers. In certain embodiments, the composite has a particular orientation.

The term “contacting” refers to any method of providing or delivering a scaffold on to or near tissue to be treated. Such methods are described throughout this document, and include injection of a biodegradable scaffold on to a tissue wound and/or molding a biodegradable scaffold in a mold and then placing the molded scaffold on a tissue wound. In some embodiments contacting refers to completely covering a skin wound, and optionally the surrounding skin, with a biodegradable polyurethane scaffold. In some embodiments contacting refers to placing a biodegradable polyurethane scaffold between two or more bone fragments that have fractured. In various aspects, a scaffold can be contact an existing tissue wound, and in further various aspects a polyurethane scaffold can be contacted prophylactically; that is, to prevent a wound from forming on tissue.

The term “nontoxic” is used herein to refer to substances which, upon ingestion, inhalation, or absorption through the skin by a human or animal, do not cause, either acutely or chronically, damage to living tissue, impairment of the central nervous system, severe illness or death.

The term “osteoconductive” as used herein, refers to the ability of a substance or material to provide surfaces which are receptive to the growth of new bone.

The term “osteogenic” as used herein, refers to the ability of a substance or material that can induce bone formation.

The term “osteoinductive” as used herein, refers to the quality of being able to recruit cells (e.g., osteoblasts) from the host that have the potential to stimulate new bone formation. In general, osteoinductive materials are capable of inducing heterotopic ossification, that is, bone formation in extraskeletal soft tissues (e.g., muscle).

The term “osteoimplant” is used herein in its broadest sense and is not intended to be limited to any particular shapes, sizes, configurations, compositions, or applications. Osteoimplant refers to any device or material for implantation that aids or augments bone formation or healing. Osteoimplants are often applied at a bone defect site, e.g., one resulting from injury, defect brought about during the course of surgery, infection, malignancy, inflammation, or developmental malformation. Osteoimplants can be used in a variety of orthopedic, neurosurgical, dental, and oral and maxillofacial surgical procedures such as the repair of simple and compound fractures and non-unions, external, and internal fixations, joint reconstructions such as arthrodesis, general arthroplasty, deficit filling, disectomy, laminectomy, anterior cerival and thoracic operations, spinal fusions, etc.

The term “porogen” as used herein, refers to a chemical compound that may be part of the inventive composite and upon implantation/injection or prior to implantation/injection diffuses, dissolves, and/or degrades to leave a pore in the osteoimplant composite. A porogen may be introduced into the composite during manufacture, during preparation of the composite (e.g., in the operating room), or after implantation/injection. A porogen essentially reserves space in the composite while the composite is being molded but once the composite is implanted the porogen diffuses, dissolves, or degrades, thereby inducing porosity into the composite. In this way porogens provide latent pores. In certain embodiments, the porogen may be leached out of the composite before implantation/injection. This resulting porosity of the implant generated during manufacture or after implantation/injection (i.e., “latent porosity”) is thought to allow infiltration by cells, bone formation, bone remodeling, osteoinduction, osteoconduction, and/or faster degradation of the osteoimplant. A porogen may be a gas (e.g., carbon dioxide, nitrogen, or other inert gas), liquid (e.g., water, biological fluid), or solid. Porogens are typically water soluble such as salts, sugars (e.g., sugar alcohols), polysaccharides (e.g., dextran (poly(dextrose)), water soluble small molecules, etc. Porogens can also be natural or synthetic polymers, oligomers, or monomers that are water soluble or degrade quickly under physiological conditions. Exemplary polymers include polyethylene glycol, poly(vinylpyrollidone), pullulan, poly(glycolide), poly(lactide), poly(lactide-co-glycolide), other polyesters, and starches. In certain embodiments, bone particles utilized in provided composites or compositions may act as porogens. For example, osteoclasts resorb allograft and make pores in composites.

In some embodiments, porogens may refer to a blowing agent (i.e., an agent that participates in a chemical reaction to generate a gas). Water may act as such a blowing agent or porogen.

The term “porosity” as used herein, refers to the average amount of non-solid space contained in a material (e.g., a composite of the present invention). Such space is considered void of volume even if it contains a substance that is liquid at ambient or physiological temperature, e.g., 0.5° C. to 50° C. Porosity or void volume of a composite can be defined as the ratio of the total volume of the pores (i.e., void volume) in the material to the overall volume of composites. In some embodiments, porosity (□□, defined as the volume fraction pores, can be calculated from composite foam density, which can be measured gravimetrically. Porosity may in certain embodiments refer to “latent porosity” wherein pores are only formed upon diffusion, dissolution, or degradation of a material occupying the pores. In such an instance, pores may be formed after implantation/injection. It will be appreciated by these of ordinary skill in the art that the porosity of a provided composite or composition may change over time, in some embodiments, after implantation/injection (e.g., after leaching of a porogen, when osteoclasts resorbing allograft bone, etc.). For the purpose of the present disclosure, implantation/injection may be considered to be “time zero” (To).

The term “remodeling” as used herein, describes the process by which native bone, processed bone allograft, whole bone sections employed as grafts, and/or other bony tissues are replaced with new cell-containing host bone tissue by the action of osteoclasts and osteoblasts. Remodeling also describes the process by which non-bony native tissue and tissue grafts are removed and replaced with new, cell-containing tissue in vivo. Remodeling also describes how inorganic materials (e.g., calcium-phosphate materials, such as β-tricalcium phosphate) are replaced with living bone.

The term “scaffold” as used herein refers to a substance that can be used to treat tissue and/or a wound. In some embodiments the scaffold or graft is a foam that can be injected between fractured bone fragments to help heal the fracture. In some embodiments the scaffold or graft is a material that can be placed on or near tissue to be treated. The terms “composite”, “scaffold”, and “graft” may be used interchangeably herein to refer to embodiments of the presently-disclosed subject matter.

The term “setting time” as used herein, is approximated by the tack-free time (TFT), which is defined as the time at which the material could be touched with a spatula with no adhesion of the spatula to the foam. At the TFT, the wound could be closed without altering the properties of the material.

The term “shaped” as used herein, is intended to characterize a material (e.g., composite) or an osteoimplant refers to a material or osteoimplant of a determined or regular form or configuration in contrast to an indeterminate or vague form or configuration (as in the case of a lump or other solid matrix of special form). Materials may be shaped into any shape, configuration, or size. For example, materials can be shaped as sheets, blocks, plates, disks, cones, pins, screws, tubes, teeth, bones, portions of bones, wedges, cylinders, threaded cylinders, and the like, as well as more complex geometric configurations.

The term “small molecule” as used herein, is used to refer to molecules, whether naturally-occurring or artificially created (e.g., via chemical synthesis), that have a relatively low molecular weight. In some embodiments, small molecules have a molecular weight of less than about 2,500 g/mol, for example, less than 1000 g/mol. In certain embodiments, small molecules are biologically active in that they produce a local or systemic effect in animals, such as mammals, e.g., humans. In certain embodiments, a small molecule is a drug. In certain embodiments, though not necessarily, a drug is one that has already been deemed safe and effective for use by an appropriate governmental agency or body (e.g., the U.S. Food and Drug Administration).

The terms “subject” or “subject in need thereof” refer to a target of administration, which optionally displays symptoms related to a particular disease, pathological condition, disorder, or the like. The subject of the herein disclosed methods can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

The term “tissue” is used herein to refer to a population of cells, generally consisting of cells of the same kind that perform the same or similar functions. The types of cells that make the tissue are not limited. In some embodiments tissue is part of a living organism, and in some embodiments tissue is tissue excised from a living organism or artificial tissue. In some embodiments tissue can be part of skin, bone, an organ or the like.

The terms “treatment” or “treating” refer to the medical management of a patient with the intent to heal, cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. For example, in some embodiments treatment refers to the healing bone tissue that is fractured and/or healing wounded skin tissue.

The term “working time” as used herein, is defined in the ISO9917 standard as “the period of time, measured from the start of mixing, during which it is possible to manipulate a dental material without an adverse effect on its properties” (Clarkin et al., J Mater Sci: Mater Med 2009; 20:1563-1570). In some embodiments, the working time for a two-component polyurethane is determined by the gel point, the time at which the crosslink density of the polymer network is sufficiently high that the material gels and no longer flows. According to the present invention, the working time is measured by loading the syringe with the reactive composite and injecting <0.25 ml every 30 s. The working time is noted as the time at which the material was more difficult to inject, indicating a significant change in viscosity.

The term “wound” as used herein refers to any defect, injury, disorder, damage, or the like of tissue. In some embodiments a wound can be a bone fracture. In some embodiments a wound is damaged skin or skin that must heal from a particular disorder.

### BRIEF DESCRIPTION OF THE DRAWINGS

FIGS. 1A-H show MasterGraft ceramic granules remodel in femoral condyle plug defects (11×18 mm) in sheep. Images of 2D (A) μCT and (B) histological sections at 2 years show new formation (NB) and <10% residual ceramic (arrows). (C) High-magnification (100×) images of histological sections at 4 mos show partial resorption (double arrows) and incorporation of the ceramic particles (arrow) in new bone. (D) Synthesis of PEUR/ceramic composite foams (20% ceramic) from LTI-PEG prepolymer, polyester (PE) triol, and TEDA catalyst. (E) Low- and (F) high-magnification images of PEUR/ceramic composites show resorption (double arrow) and incorporation of the ceramic particles (arrow) in new bone, as well as lamellar bone (LB) formation on the new bone surface at 4 mos. Histomorphometric analysis of PEUR/ceramic and ceramic groups at 12 months shows (G) 25-40% new bone increasing and (H) almost complete resorption of the ceramic.

FIG. 2 shows a schematic view of the synthesis of a thioketal (TK) diol.

FIG. 3 shows a schematic view of the synthesis of a nHA-LTI prepolymer (0-65 wt % nHA).

FIGS. 4A-G show graphs and images illustrating synthesis of an nHA-lysine triisocyanate (LTI) prepolymer. (A) shows a schematic of LTI being grafter to the surface of nHA through reaction of the two primary NCO groups with P—OH groups on the surface of nHA. (B) shows a graph illustrating the measured value of the % NCO of the catalyzed mixture (open black circles), the theoretical values of the % NCO of the catalyzed mixture (filled black circles), and the experimental NCO content (blue line), which confirms the presence of grafted LTI. (C) shows a graph illustrating FTIR analysis of catalyzed nHA/LTI mixture. (D) shows graphs illustrating the resulting increases in the N is and C is peaks for nHA-LTI compared to nHA from the grafting of LTI to nHA. (E) shows graphs illustrating individual peak analysis for nHA and nHA-LTI. (F) shows graphs and images illustrating particle size distribution for nHA and nHA-LTI. (G) shows a graph illustrating crystallinity for nHA and nHA-LTI.

FIG. 5 is a schematic view of the synthesis of an nHA-PTKUR inorganic-organic hybrid polymer network. Black circles represent nHA particles (shown bound to 4 LTI molecules for clarity of presentation), gray circles represent urethane (—O—C═O—NH—) bonds, TK represents thioketal residues, and Lys represents lysine residues.

FIGS. 6A-F show synthesis of viscous nHA-LTI prepolymers (≤65 wt % nHA). (A) nHA (Ca₅(PO₄)₃OH) is reacted with LTI at an NCO:OH ratio ≥3 (≤65 wt % nHA). (B) shows a graph illustrating phase change and particle size distribution for nHA and nHA-LTI after adding a catalyst and mixing. (C) SEM images of nHA powder and nHA-LTI particles recovered from the nHA-LTI prepolymer show that the majority of the particles are <100 nm (yellow line). (D) viscosity of nHA-LTI (40 and 65 wt % nHA) prepolymers versus shear rate. (E) compressive strength increases with nHA content in the prepolymer. Strength of nHA-PTKUR hybrid polymers with nHA ≥20 wt % exceeds that of PEUR/nHA composites (nHA added as a powder) incorporating 52 wt % nHA. (F) shows a graph illustrating FTIR spectra, which showed a reduction in the NCO peak area in the presence of the catalyst.

FIGS. 7A-H show bone cell activity and in vivo remodeling of PEUR/nHA composites. Expression of (A) Runx2 and (B) Opn by mouse MC3T3 cells is higher for PEUR/nHA composites compared to PEUR. (C) At day 21, deposition of mineralized bone matrix (white arrows, assessed by Alizarin Red staining) is higher on PEUR/nHA compared to PEUR. (D) When co-cultured with MSCs on PEUR/nHA composites, RAW 264 monocytes differentiate to form osteoclasts that create resorption pits (white arrows, day 21). (E-H) In vivo remodeling of PEUR/nHA composites injected into 6×11 rabbit femoral condyle plug defects at (E-F) 6 and (G-H) 12 weeks. Ingrowth of new bone into the composite is indicated by the white arrows.

FIG. 8 shows safety phosgenation process for manufacture of LTI. (A) Synthesis of lysine ester trihydrochloride salt. (B) Catalytic decomposition of triphosgene to phosgene. (C) Synthesis of LTI from the trihydrochloride salt and phosgene. (D-F) Photographhs of filled (D) syringes, (E) foil pouches, and (F) foil laminate tubes. (G) % NCO and (H) working time of PEG-LTI prepolymer stored at 60° C. for up to 8 weeks.

FIGS. 9A-I show graphs and images illustrating formation and properties of nHA and nHA-LTI cements. (A) shows a graph illustrating decreasing viscosity with increasing shear rate. (B) shows an image illustrating a double-barrel syringe fitted with a static mixer. (C) shows a schematic illustrating formation of a crosslinked organic-inorganic hybrid cements. (D) shows SEM images illustrating dispersion of nHA and nHA-LTI in the cement. (E) shows a graph illustrating the area percentage of nHA-LTI aggregates as compared to nHA aggregates. (F) shows a graph illustrating the effects of LTI grafting and increasing isocyanate index on swelling. (G) shows an image illustrating measurement of four-point bending properties of nHA and nHA-LTI. (H) shows graphs illustrating increased cement bending modulus and bending strength in surface grafting versus no grafting. (I) shows graphs illustrating yield strength of nHA-LTI and nHA cements.

FIGS. 10A-H show graphs and images illustrating the effects of nHA-LTI grafting on the properties of the cements. (A) shows a graph illustrating the effect of nHA-LTI grafting on water contact angle. (B) shows a graph illustrating the effect of nHA-LTI grafting on protein adsorption of fibronectin and vitronectin. (C) shows an image illustrating MC3T3 pre-osteoblast cell death 48 hours after cell seeding. (D) shows a graph illustrating total protein increase. (E) shows a graph illustrating cell proliferation of cells seeded on different cements. (F) shows images illustrating mineralization as assessed by Alizarin Red staining. (G) shows a graph illustrating quantification of staining by extraction of Alizarin Red from the substrates. (H) shows images illustrating the area % of stained surface.

FIG. 11 shows an image illustrating a sagittal view of tibial plateau and femoral plug defects in sheep.

### DESCRIPTION OF THE INVENTION

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes nanocrystalline hydroxyapatite (nHA)/polyurethane (PUR) hybrid composites. According to one or more of the embodiments disclosed herein, the hybrid composites enhance bone cell activity, exhibit bone-like strength, and/or are hydrolytically stable but cell-degradable. In one embodiment, the hybrid composites include (nHA)-polyurethanes such as, but not limited to, nHA-(thioketal urethane)s (PTKUR)s, nHA-poly(ester urethane)s, lysine-derived polyurethanes, any other suitable polyurethane, and/or a combination thereof. In another embodiment, the (nHA)-polyurethanes are inorganic-organic hybrid polymers. In a further embodiment, the composites are hydrolytically stable and undergo cell-meditated oxidation of the lysine and thioketal (TK) residues while nHA is resorbed by osteoclasts. In still a further embodiment, the composites/polymers degrade to non-cytotoxic breakdown products and/or can be manufactured at the kilogram scale. In contrast to biphasic poly(ester urethane) (PEUR)/ceramic composites, which degrade by hydrolysis before cells fully infiltrate a graft and/or comprise apatitic cements, polymers, and/or degradable metals with limited biological activity, the hydrolytically stable PTKUR composite includes polymer and ceramic resorption rates that are balanced with new bone formation rates.

As graft resorption and new bone formation are regulated by endogenous cells, both processes are aligned with patient biology in the hybrid polymers described herein. Without wishing to be bound by theory, it is believed that the balanced resorption and bone formation rates decrease or eliminate resorption gaps and/or fibrous scar formation. For example, in some embodiments, the hybrid polymer supports new bone formation, persists throughout the bone remodeling phase, and resorbs almost completely at 2 years. Additionally, in some embodiments, reinforcement of calcium phosphate cements with polymer or metal fibers increases the toughness of the material by up to two orders of magnitude. Furthermore, and once again without wishing to be bound by theory, it is believed that nHA in the hybrid polymer stimulates new bone formation by enhancing differentiation of local osteoprogenitor cells to osteoblasts. For example, in one embodiment, the ceramic forms a dispersed particulated phase that provides an osteoconductive scaffold for new bone formation as the polymer binder degrades and cells infiltrate the composite. In another embodiment, the hybrid composites improve healing of large bone defects by enhancing osteogenic differentiation of endogenous cells, providing bone-like strength, and/or aligning graft resorption with patient biology. In a further embodiment, since such composites can be readily mixed with osteobiologics or anti-microbials at the point of care, nHA-PTKUR BVFs also facilitate repair of open fractures of the tibia or mandible >3 cm in length.

In some embodiments, varying the amount of inorganic component in the hybrid polymers modifies the mechanical properties and/or biological properties. For example, in one embodiment, mechanical properties, osteoblast differentiation, and new bone formation increase with nHA loading. In another embodiment, nHA loading in the nHA-PTKUR hybrid polymers enhance osteoblast differentiation, new bone formation, and mechanical properties in a dose-responsive manner (i.e., will increase with nHA loading). In a further embodiment, the hybrid polymers contain, by weight percent of nHA into LTI, up to about 65%, between about 1 and about 65%, between about 5 and about 65%, between about 10 and about 65%, between about 15 and about 65%, between about 20 and about 65%, between about 30 and about 65%, between about 40 and about 65%, or any combination, sub-combination, range, or sub-range thereof. In one embodiment, the hybrid polymers having up to 65 wt % nHA into LTI provide a liquid, reactive prepolymer that may further react and blend with esters and inorganic phase. This increases the amount of ceramic component in the hybrid material dramatically, which, in some embodiments, enhances mechanical properties and bio-reactivity of the hybrid material.

Additionally or alternatively, the hybrid composites/polymers may include one or more additives. For example, in one embodiment, the one or more additives include one or more granular particles having a size of between about 100 and about 500 microns. In another embodiment, the granular particles enhance handling properties of the materials. In a further embodiment, the type and/or amount of the one or more additives is selected to provide one or more desired properties. Suitable granular particles include, but are not limited to, porogens and/or ceramic particles.

In certain embodiments, the granular particles may transform an injectable to a solid or substantially solid putty. For example, an injectable, flowable composite and/or cement may be formed from nHA and polyurethane alone, otherwise being devoid or substantially devoid of additives such as granular particles. The addition of granular particles, however, may transform that injectable, flowable composite into a moldable structural composite. In some embodiments, a moldable structural composite and/or cement may be desired, such as, for example, for treatment of weight-bearing bone defects. While the amount of additive in the composite may vary based upon the formulation, in one embodiment, a suitable amount of porogen includes between 0 and 50 wt %, between 1 and 50 wt %, between 5 and 50 wt %, between 0 and 45 wt %, between 0 and 40 wt %, between 0 and 30 wt %, between 0 and 20 wt %, between 0 and 15 wt %, between 5 and 20 wt %, between 5 and 15 wt %, between 0 and 10 wt %, between 5 and 10 wt %, or any combination, sub-combination, range, or sub-range thereof. Additionally or alternatively, a suitable amount of ceramic, such as MasterGraft, includes between 3 and 50 wt %, between 5 and 50 wt %, between 10 and 50 wt %, between 15 and 50 wt %, between 20 and 50 wt %, between 20 and 45 wt %, between 20 and 40 wt %, or any combination, sub-combination, range, or sub-range thereof.

For example, the composite may include up to 45 wt % sucrose particles (porogen), up to 10 wt % MasterGraft ceramic and up to 35 wt % sucrose porogen, up to 45 wt % MasterGraft ceramic, or any suitable combination, sub-combination, range, or sub-range thereof. In the case of a high porosity bone void filler, the composite may include more porogen than ceramic. For example, in certain embodiment, a high porosity bone void filler may include between 0 and 20 wt % MasterGraft and between 20 and 50 wt % porogen.

In some embodiments, the hybrid composites form injectable and settable bone void fillers. These injectable bone void fillers may be used to fill bony voids in the skeletal system. For example, the bone void fillers may be used to fill bony voids of up to, equal to, and/or greater than 3 cm, including, but not limited to, large metaphyseal bone defects. Such hybrid composites increase strength, enhance osteogenic differentiation of endogenous cells, increase mechanical stability, align graft resorption with patient biology, and/or allow for cellular infiltration into the graft. Additionally or alternatively, in some embodiments, the hybrid composite forms a moldable nHA-PTKUR/ceramic granule (CG) composite bone void filler. In certain embodiments, slowly-degrading ceramic granules (CG, 100-300 μm) facilitate osseointegration by acting as a scaffold over which bone can grow.

The presently-disclosed subject matter also includes a method of forming Poly(thioketal urethane) (PTKUR)/ceramic composites. In some embodiments, viscous nHA-lysine triisocyanate (LTI) prepolymers can be made in one step without the use of solvents. For example, in one embodiment, nHA particles are reacted with lysine derived triisocyanate (LTI) at a NCO:OH ratio of 3:1, to form a nHA/LTI hybrid prepolymer (65 wt % n-HA). The nHA may be provided from any suitable source, including, but not limited to, Nanostim™ Resorbable nHA Bone Paste (<20 nm, Medtronic). In another embodiment, nHA-LTI prepolymers can be synthesized with NCO:OH ratios varying from about 20:1 to 3:1 (20-65 wt % nHA) and crosslinked with a thioketal (TK) diol to form nHA-PTKUR inorganic-organic polymer networks. The thioketal (TK) diol crosslinker is designed to be hydrolytically stable but oxidatively degradable. Additionally, the TK diol has thioketal bonds that are destabilized by hydroxyl radicals, resulting in chain scission and breakdown to the original monomers with a minimal inflammatory response.

The nHA/LTI mixture described herein has a texture similar to wet sand at first, and after 5 hours of reaction is turned into a viscous liquid. The resulting prepolymer may be further reacted with polyester to form a tough nHA/PUR hybrid polymer network (55% n-HA). For example, an LTI-TK prepolymer may be mixed with the TK diol, ceramic particles, and an iron acetylamide catalyst with a high selectivity for the gelling reaction to form a low-porosity PTKUR/ceramic composite cement. The PTKUR polymer formed according to one or more of the embodiments disclosed herein is hydrolytically stable but degrades in oxidative medium simulating the reactive oxygen species (ROS) secreted by adherent cells.

In certain embodiments, the nHA particles include a grain size of less than 100 nm. Such grain sizes enhance attachment, proliferation, and osteogenic differentiation of endogenous precursor cells; osteoclast differentiation and activity; and new bone formation. In one embodiment, nHA particles specific surface of greater than 10 m² g⁻¹ enhances interfacial bonding with the polymer due to their increased surface area and reactivity, resulting in higher mechanical properties. In another embodiment, replacement of hydrolytically labile PEUR with hydrolytically stable PTKUR maintains mechanical stability in the interior of the graft prior to remodeling by inhibiting hydrolysis of the polymer at late stages of healing. Thus, in a further embodiment, nHA-PTKUR/ceramic composites exhibit strength exceeding that of bone, enhance new bone formation, and align the rates of graft resorption and healing.

As discussed in detail above, the Nanohydroxyapatite (nHA)-polyurethane hybrid inorganic-organic polymers described herein exhibit enhanced mechanical properties compared to polyurethane alone. In some embodiments, the hydroxyl (P—OH) group on the surface of nHA is reactive and may be used to graft organic molecules, including polyisocyanates. Additionally or alternatively, covalently bonding nHA to the polyurethane to form an inorganic-organic hybrid polymer network enhances nHA dispersion and mechanical properties relative to embedding nHA in the polymer. In some embodiments, for example, the composites exhibit compressive strengths exceeding that of trabecular bone and calcium phosphate cements, with yield strength 47.0±13.4 MPa. In certain embodiments, the composites undergo cell-mediated oxidative degradation and remodel in bone defects. For example, when injected into 6×11 mm defects in the femoral condyle of New Zealand White rabbits, PTKUR/ceramic composites showed densification of the host bone near the surface of the composite, as well as ingrowth of new trabaculae near the interface, which was also observed for the ceramic particles control. Thus, PTKUR/ceramic composites exhibit evidence of remodeling near the host bone/composite interface at this early time point.

Unlike existing formulations of MasterGraft® which do not set and harden to form grafts with bone-like strength, the composites/polymers described herein combine a reactive nHA-PTKUR inorganic-organic hybrid polymer with MasterGraft Mini Granules to create an injectable, settable, and resorbable bone void filler with initial bone-like strength. More specifically, and without wishing to be bound by theory, the nHA component is believed to enhance the biological and mechanical properties of the composite, the cell-degradable PTKUR component is believed to align polymer resorption with patient biology, and the MasterGraft is believed to serve as a scaffold for bone growth. In some embodiments, the bone-like strength of the nHA/PTKUR BVFs improve articular reducation and joint stability, thereby improving outcomes in intra-articular fractures. In addition to use as a bone void filler (BVF), other uses of the composite/polymer described herein include injectable and resorbable bone graft for treating open fractures at weight-bearing sites due to its favorable handling, mechanical, and biological properties. Further uses of the composite/polymer include, but are not limited to, augmenting the nHA-PTKUR BVFs with biologics at the point of care to enhance healing of open fractures.

The presently-disclosed subject matter also includes packaging and method of sterilizing the packaging. In one embodiment, the packaging includes long-term packaging in cyclic olefin polymer syringes (outgassed with N₂ to eliminate urea formation observed previously), (foil laminate pouches), and/or foil laminate tubes. In some embodiments, based on the polymer class, the sterilization includes radiation, such as, but not limited to, gamma or e-beam sterilization methods.

### EXAMPLES

The presently-disclosed subject matter is further illustrated by the following specific but non-limiting examples. Some examples are prophetic. Some examples may include compilations of data that are representative of data gathered at various times during the course of development and experimentation related to the presently-disclosed subject matter.

### Example 1— Analysis of New Bone Formation, Remodeling, and Resorption

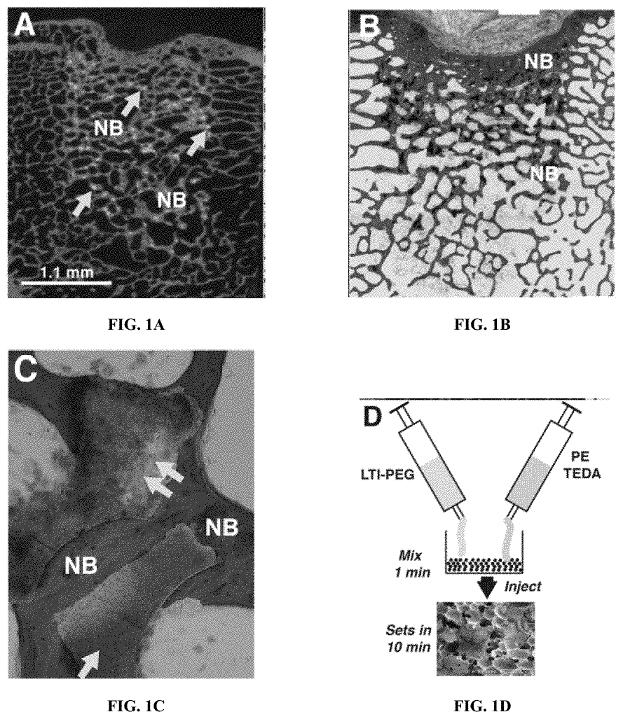

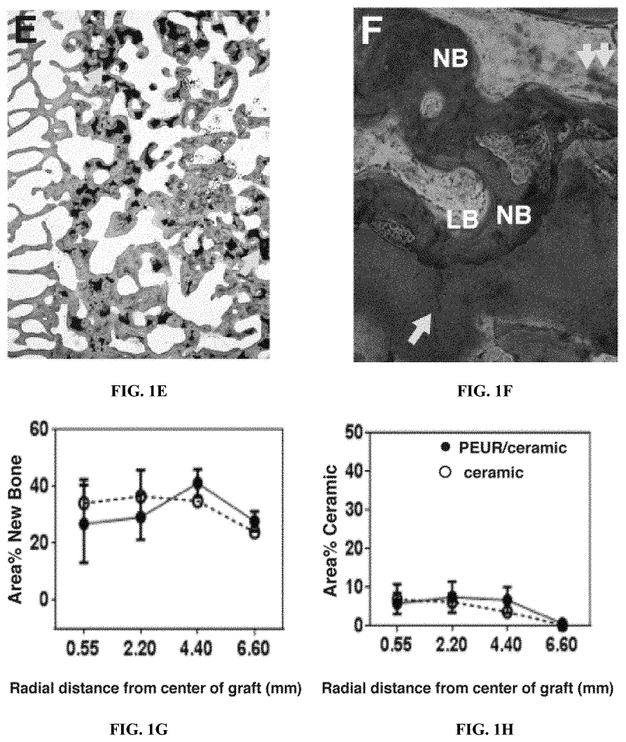

This example illustrates that ceramic particles comprising about 15% hydroxyapatite/85% β-TCP (MasterGraft, Medtronic) support new bone formation, persist throughout the bone remodeling phase, and resorb almost completely at 2 years (FIG. 1A-B). High (100×) magnification views at 4 months show incorporation of the ceramic particles within the bone, as well as partial resorption of the ceramic (FIG. 1C). Bone healing was similar for poly(ester urethane) (PEUR)/ceramic composite foams prepared by mixing a lysine triisocyanate (LTI)-polyethylene glycol (PEG) prepolymer with a polyester (PE) triol and triethylenediamine (TEDA) catalyst (FIG. 1D). The material expanded by about 50% due to the CO₂-generating reaction of LTI-PEG with water. Similar to the ceramic particles alone, partial resorption and incorporation of the ceramic in new bone were observed at 4 months (FIG. 1E-F). Histomorphometric analysis at 12 months revealed that for both the ceramic and PEUR/ceramic groups, new bone formation ranged from 25-40% and residual ceramic was <8%. However, healing in the PEUR/ceramic groups was more variable due to the lower (˜20 vol %) ceramic content as well as hydrolytic degradation of the poly(ester urethane) in the interior of the graft. Furthermore, the PEUR/ceramic foams had low strength (˜1 MPa). Taken together, these data show that PEUR/ceramic composites remodel in a large animal model of bone regeneration, and point to increased ceramic loading and hydrolytically stable, cell-degradable polymers as effective approaches for enhancing bone healing.

### Example 2—Synthesis of Lysine Trihydrochloride Salt

Ethanolamine hydrochloride (124 g) is placed into a flask fitted with mechanical stirrer, thermocouple, gas inlet tube and vacuum fitting and heated to 90° C. to form a melt. Lysine mono-hydrochloride (101 g) is added to the melt to maintain a free-flowing slurry. After the addition is complete, a vacuum is established over the reaction mixture and the temperature increased to 120° C. while HCl gas is bubbled into the reaction mixture (˜5-10 ml/min for 5 hours). When the disappearance of lysine by ¹H NMR is observed, the mixture is cooled to 90° C. and diluted with methanol (0.5 l) to yield a solution that may be further diluted with denatured ethanol to a total volume of —1.71. Solids formed by slow cooling overnight are isolated by vacuum filtration and stored in a dessicator. The lysine trihydrochloride salt is purified by dissolving in methanol and subsequent dilution with ethanol near reflux temperature as described above. The purified product (˜65% yield) is recovered as a white crystalline solid.

### Example 3—Preparation of Phosgene/Chlorobenzene Solution

Triphosgene (10 g) is placed in a reaction flask fitted with a magnetic stir bar, expansion bulb (to control foaming), and thermocouple. 1,10-phenanthroline (50 mg) is added followed by sealing of the reactor. A tube is run to another flask containing chlorobenzene (25 g). This flask is cooled in an ice bath and fitted with a dry ice condenser vented to a NaOH scrubber. The flask containing the triphosgene is heated slowly to a maximum of 105° C. At 80° C., the triphosgene melts resulting in gas generation, which is absorbed in the chlorobenzene.

### Example 4—Lysine Triisocyanate (LTI) Synthesis

A solution of phosgene (97 g) in chlorobenzene (220 g) is first prepared. Lysine ester trihydrochloride salt (50 g) is then charged to a flask fitted with a mechanical stirrer, thermocouple, and dry ice condenser. The reactor outlet is attached to a scrubber, chlorobenzene (0.5 l) is charged and a suspension formed. The mixture is heated to 120° C. and the phosgene solution subsequently added slowly via pump (˜10 ml/min). Addition of the phosgene is controlled to maintain a reaction temperature above 115° C. The reaction is heated for 11 h to reach complete conversion to LTI by ¹H NMR analysis.

### Example 5—LTI Purification

The oil is placed on a rotary evaporator and heated to 65° C. at 0.9 mm Hg overnight to remove the chlorobenzene to <5000 ppm. The oil is then dissolved in methyl tert-butyl ether (MTBE, 10% LTI in MTBE) and decanted to remove insoluble LTI oligomers. The filtered solution is then flowed through an activated carbon bed to remove residual LTI oligomers and lysine hydrochloride. Adsorption isotherms and breakthrough curves are measured for varying flow rates and bed lengths. Strong acid cation exchange resins (e.g., Dowex HCR-W2) that have been reported to highly selective for lysine adsorption are also investigated for removal of the lysine trihydrochloride impurity.

### Example 6—TK Diol Crosslinker Synthesis

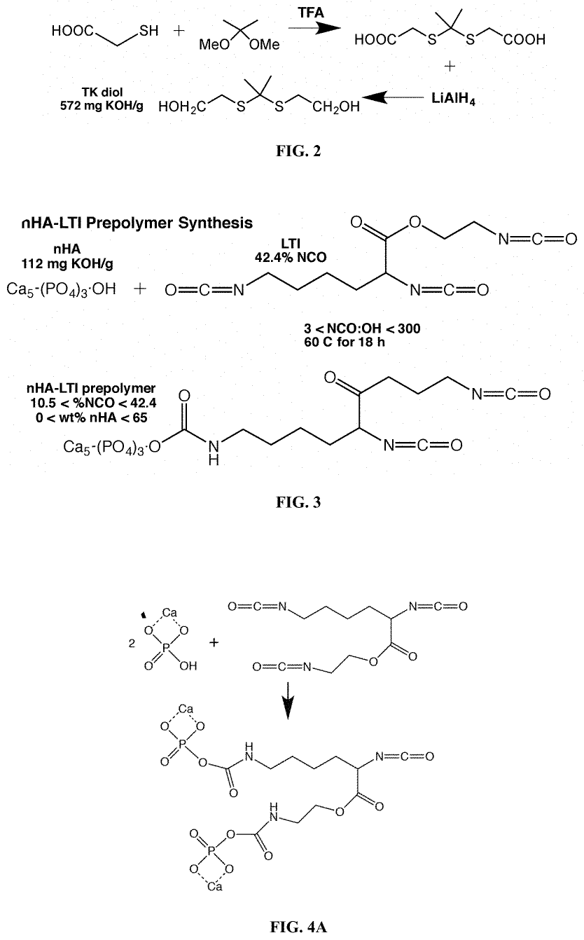

The synthesis scheme is shown in FIG. 2. Anhydrous bismuth(III) chloride (BiCl₃) catalyst is added to a round-bottom flask under anhydrous N₂. Approximately 100 ml anhydrous acetonitrile is then added to the flask, followed by 2,2-dimethoxypropane (DMP, 14.7 ml) and thioglycolic acid (TGA, 7 ml). The mixture is reacted for 24 h at room temperature, followed by rotary evaporation of acetonitrile and vacuum drying. The resulting di-acid intermediate (20.2 g) is added to a flask with LiAlH₄ catalyst (7.6 g) under N₂, then 150 ml diethyl ether solvent is added followed by 200 ml anhydrous THF (200 ml) added drop-wise at 0° C. over 2-3 h. The mixture is refluxed at 50° C. for 6 h. The reaction is then quenched with water (drop-wise), diluted in dichloromethane, filtered to remove by-products, and washed with 10% sulfuric acid solution. The organic phase in the filtrate is recovered and treated with sodium sulfate to remove residual water, filtered, rotary evaporated, and dried to yield the TK diol crosslinker. Structure may be confirmed by NMR, viscosity may be measured by rheometry, and OH Number measured by titration.

### Example 7—nHA-LTI Prepolymer Synthesis

In this example of a synthesis of nHA-LTI prepolymer, Nanostim™ Resorbable nHA Bone Paste (<20 nm, Medtronic) is used as the source of nHA particles. The reaction scheme is shown in FIG. 3. nHA (Ca₄PO₄)₃OH, OH No.=112 mg KOH/g) is reacted with LTI (42.4% NCO) to yield an nHA-LTI prepolymer overnight at 60° C. The NCO:OH ratio is varied between about 3≤NCO:OH≤300 to synthesize prepolymers varying from 0-65 wt % nHA, which data showed is the highest nHA content that yields a viscous liquid prepolymer. Prepolymers may be characterized for % NCO and reaction conversion (titration), viscosity (AR-G2 rheometer), chemical composition (FTIR), and particle size distribution (Malvern ZetaSizer). The reaction kinetics (second-order specific reaction rate) and conversion will be monitored by ATR-FTIR as the disappearance of the NCO peak (3 m⁻¹) in the IR spectrum.

### Example 8—nHA-LTI Prepolymer Synthesis and Characterization

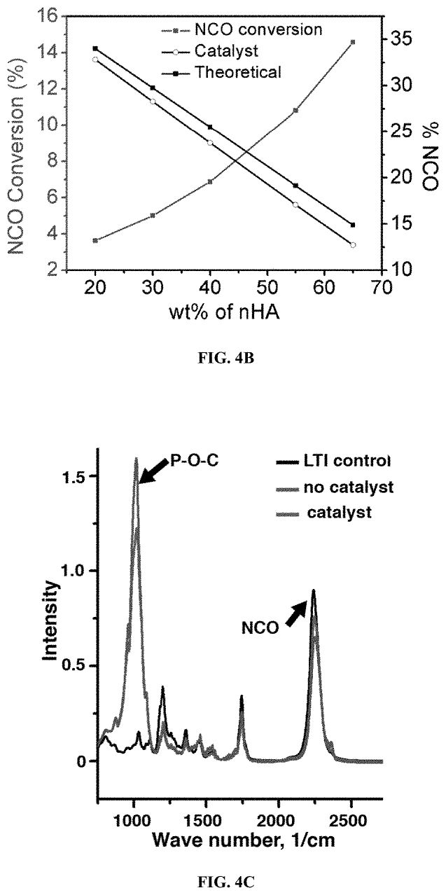

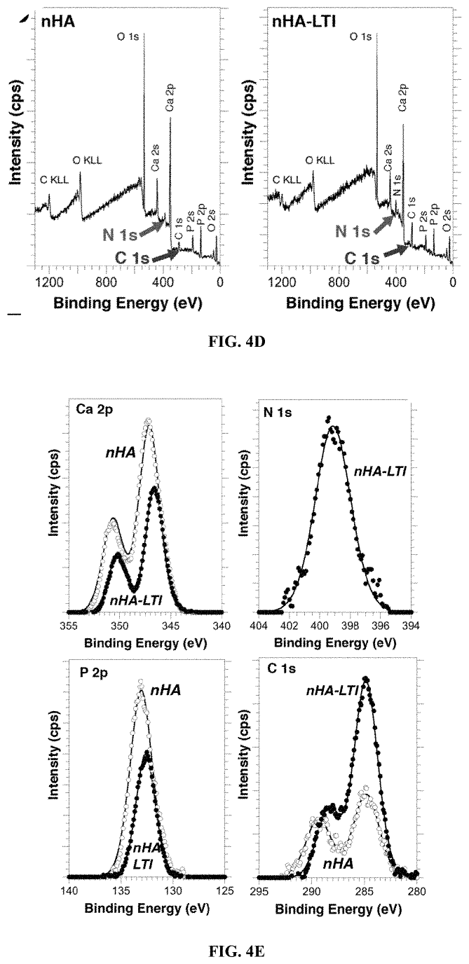

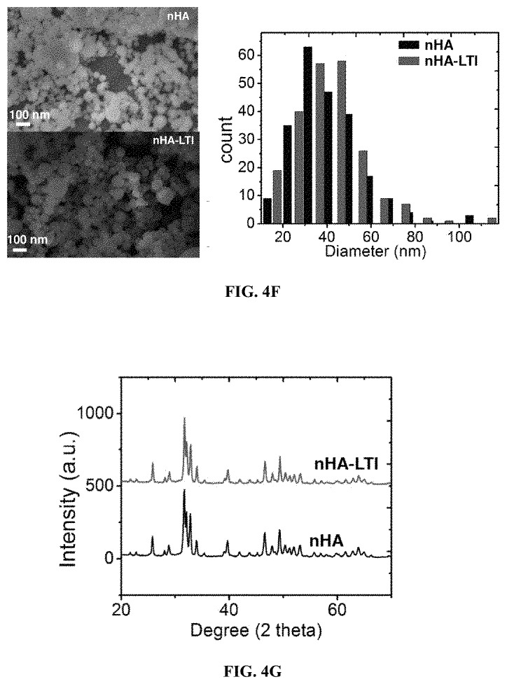

To synthesize nHA-lysine triisocyanate (LTI) prepolymer, nHA (65 wt %) and lysine triisocyanate (LTI) (35 wt %) were mixed with iron acetylacetonate (FeAA) catalyst (0.027 wt %) at 50° C. for 3 hours. The reaction mixture initially had a granular texture comparable to wet sand. After adding FeAA catalyst and mixing for 1 minute, the mixture changed from wet sand to a viscous dispersion of nHA-LTI in LTI (nHA-LTI/LTI). LTI was grafted to the surface of nHA through reaction of the two primary NCO groups with P—OH groups on the surface of nHA (FIG. 4A). To confirm the presence of grafted LTI, the % NCO of the catalyzed mixture (open black circles in FIG. 4B) was measured by titration as a function of nHA concentration. Theoretical values of % NCO (filled black circles in FIG. 4B) were calculated based on dilution assuming no reaction. The conversion of NCO groups to phosphate urethane groups is

$${ξ}_{NCO} = \frac{\% {NCO}_{0} - \% NCO}{\% {NCO}_{0}} \qquad ( 1 )$$

where % NCO₀ and % NCO represent the theoretical (calculated assuming dilution) and experimental (meaured for the catalyzed mixture) NCO content. NCO (blue line in FIG. 4B) increased from 3.6 to 14.6% with increasing nHA concentration.

FTIR analysis of catalyzed nHA/LTI mixture showed a reduction in the N═C=O peak at 2260 cm⁻¹ and increased P—O—C peak at 1140 cm⁻¹, suggesting consumption of N═C=O groups of LTI molecule and formation of new P—O—C bond in the presence of catalyst (FIG. 4C), which further confirms the reaction of LTI with nHA in the presence of catalyst.^{[17]} The nHA particles grafted with LTI (nHA-LTI) were recovered from the nHA-LTI/LTI prepolymer and conversion of OH groups on the surface of nHA was assessed by XPS. Grafting LTI to nHA resulted in an increase in the N is (red arrow FIG. 4D) and C 1s (blue arrow FIG. 4D) peaks for nHA-LTI compared to nHA. Individual peak analysis revealed shifts in the Ca 2p, N 1s, P 2p, and C is peaks in response to surface grafting (FIG. 4E). Binding energies for N is (399.1 eV) and C is (288 eV) measured for nHA-LTI confirmed the presence of urethane bonds (—COONH—), while the binding energy of P 2p (132.6 eV) is representative of the phosphate group in hydroxyapatite. Carbon (289 eV) detected on the surface of nHA was adventitious adsorbed (not covalently bound) organic material typically observed for samples exposed to air (FIG. 4E and Table 1). Quantitative analysis showed that Ca/P=1.7 for both nHA-LTI and nHA, consistent with the composition of HA, while the C:P and N:P ratios increased after surface grafting (Table 1). The conversion of OH groups (ξ_{OH}) is calculated from:

$${ξ}_{OH} = \frac{C : P}{C : {P}_{100 \%}} = \frac{N : P}{N : {P}_{100 \%}} \qquad ( 2 )$$

where C:P_{100%}=5.5 and N:P_{100%}=1.5 are the atomic ratios assuming complete reaction of the OH groups with the primary NCO groups in LTI (FIG. 4A). Thus, the conversion of OH groups was 40%.

TABLE 1

| | Ca:P | C:P | N:P |
|---|---|---|---|
| nHA | 1.7 | 0.6 | ND |
| nHA-LTI | 1.7 | 2.2 | 0.6 |

The effects of LTI grafting on nHA particle size and crystallinity were assessed by SEM and x-ray diffraction (XRD). The particle size distribution measured from SEM images (FIG. 4F) showed no difference in the mean size of nHA (45±16 nm) and nHA-LTI (45±15 nm) particles. Similarly, grafting did not alter nHA crystallinity (FIG. 4G). The grain size determined from the XRD spectra using Scherrer equation was 36 nm (0 0 2 Miller's plane family) for both nHA and nHA-LTI.

### Example 9—nHA-PTKUR Inorganic-Organic Hybrid Polymer Synthesis

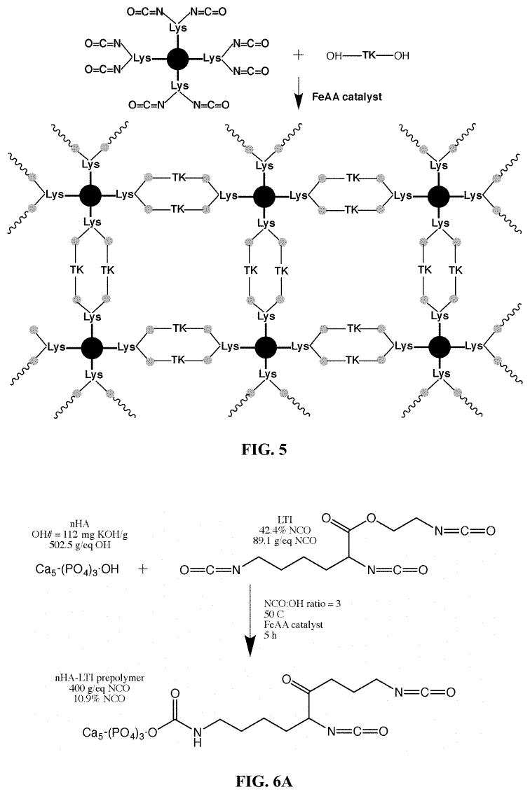

This Example shows synthesis of an nHA-PTKUR inorganic-organic hybrid polymer. The synthesis scheme is shown in FIG. 5, and the study design is listed in Table 2. The study is designed to answer the question: Does the nHA-PTKUR hybrid polymer have superior properties to PTKUR/nHA composites in which the nHA is added as a powder? Each synthesized nHA-LTI prepolymer is mixed with the TK diol at an NCO:OH ratio=1.15 in the presence of 0.5 wt % iron acetylacetonate (FeAA) catalyst. The reaction is performed at room temperature and no solvent is used. Reaction kinetics (second-order specific reaction rate) and conversion are monitored by ATR-FTIR as the disappearance in the NCO peak in the IR spectrum (2230 cm⁻¹). The working and setting times are determined as the intersection of the G′ (storage) and G″ (loss) moduli (AR-G2 rheometer). Specimens for compression, bending, and dynamic mechanical testing can be incubated in PBS at 37° C. for 24 h prior to testing. Dynamic mechanical properties (E′, E″, and tan δ) of 13.5 mm×25 mm×2 mm rectangular specimens are measured in 3-point bending mode (TA Instruments Q800 DMA). Both frequency (0.1-10 Hz) and temperature (−50-150° C.) sweeps are performed.

For compression testing, each cylindrical compression specimen (6 mm D×12 mm H) can be loaded at 25 mm/min by the platens of a material testing system (Bionix 858, MTS). The modulus of elasticity, yield strength, and energy uptake can be determined using ASTM 695-96. Additionally, the bending strength and modulus of elasticity can be determined from 4-point bending tests using 40 mm×4 mm×2 mm slabs (ISO 5833). X-ray diffraction (XRD) is performed on a Scintag X₁ θ/θ automated powder X-ray diffractometer in the range of 15-50 in 2 theta using a Cu Kα radiation source and a zero-background Si(510) sample support. Composite morphology is assessed by SEM. Samples are sputter-coated with gold and images obtained using a Hitachi S-4200 SEM and processed using the Quartz PCI system software. Thermal transitions will be assessed by Differential Scanning calorimetry (TA Instruments Q1000 DSC).

TABLE 2

| Treatment Group | nHA wt % | Replicates |
|---|---|---|
| PTKUR (control) | 0 | 4 |
| nHA(25)-PTKUR | 25 | 4 |
| nHA(45)-PTKUR | 45 | 4 |
| nHA(65)-PTKUR | 65 | 4 |
| PTKUR/nHA(25) | 25 | 4 |
| PTKUR/nHA(45) | 45 | 4 |
| PTKUR/nHA(65) | 65 | 4 |

Effects of nHA loading on properties of nHA-PTKUR hybrid polymers. These materials will used for evaluation of physical, mechanical, and biological properties. For PTKUR/nHA composites, nHA powder will be added to the reactive mixture without the previous step of synthesizing a prepolymer.

### Example 10—nHA-LTI Prepolymers Used in Preparation of Polymers

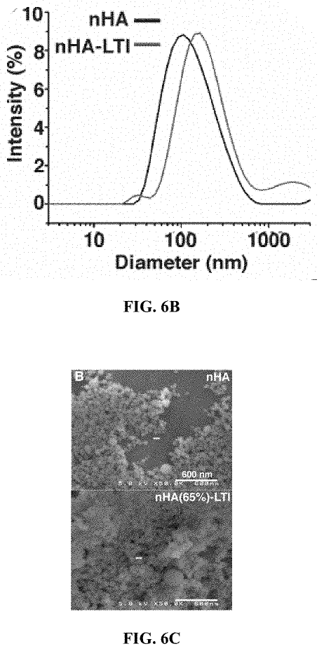

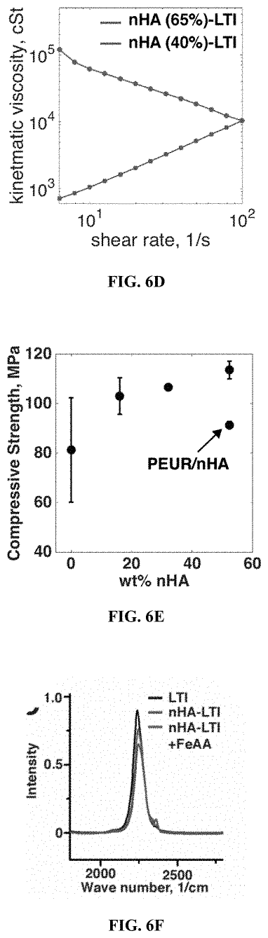

In this example, nHA-LTI prepolymers are used to prepare nHA-polyurethane hybrid inorganic-organic polymers incorporating up to 52 wt % nHA. nHA-lysine triisocyanate (LTI) prepolymers were synthesized by reacting nanocrystalline hydroxyapatite (nHA, 19.5 m²/g, 100 nm, SigmaAldrich) particles with LTI at an NCO:OH ratio ≥3:1 (FIG. 6A). The reaction mixture (65 wt % nHA) initially had a granular texture comparable to wet sand, but after adding the catalyst and mixing for 5 min it the phase changed from a solid to an opaque viscous liquid with a particle size distribution comparable to that of unreacted nHA (FIG. 6B). SEM images show that the majority of the nHA-LTI particles remained <100 nm after the LTI reaction (FIG. 6C).

The viscosity of nHA-LTI prepolymers was measured as a function of shear rate for prepolymers incorporating 40 and 65 wt % nHA (FIG. 6D). The 40 wt % prepolymer was shear-thickening and exhibited kinematic viscosity <1000 cSt at 1 s⁻¹, which is an order of magnitude lower than that of the PEG-LTI³⁵ and TK-LTI prepolymers (20,000 cSt). The 65 wt % nHA prepolymer was shear-thinning and at low shear exhibited kinematic viscosity >100,000 cSt, which is approaching the limit of injectability. nHA-LTI prepolymer (0-65 wt % nHA) was crosslinked with poly(ε-caprolactone) (PCL) triol⁴ to yield tough nHA-PEUR hybrid polymer networks (0-52 wt % nHA). Yield strength increased with nHA loading up to 52 wt %, and nHA-PEUR hybrid polymers with >20 wt % nHA exhibited higher compressive strength than PEUR/nHA composites (nHA added as a powder with no separate prepolymer step) with 52 wt % nHA (FIG. 6E). Thus, nHA-LTI prepolymers with <65 wt % nHA are useful for synthesizing hybrid inorganic-organic polymers with enhanced strength compared to bi-phasic composites. Based on % NCO measurements of LTI and the nHA-LTI prepolymer, the conversion of hydroxyl groups on the surface was >90% after reacting in the presence of FeAA catalyst for 5 h. The reaction was further confirmed by FTIR spectra, which showed a reduction in the NCO peak area in the presence of the catalyst (FIG. 6F). The nHA-LTI prepolymer was further reacted with poly(ε-caprolactone) triol to yield a tough nHA-PEUR hybrid polymer network (55 wt % nHA) OF nHA-LTI/PEUR.

### Example 11—nHA Enhances Osteogenic and Osteoclastogenic Differentiation of Endogenous Cells

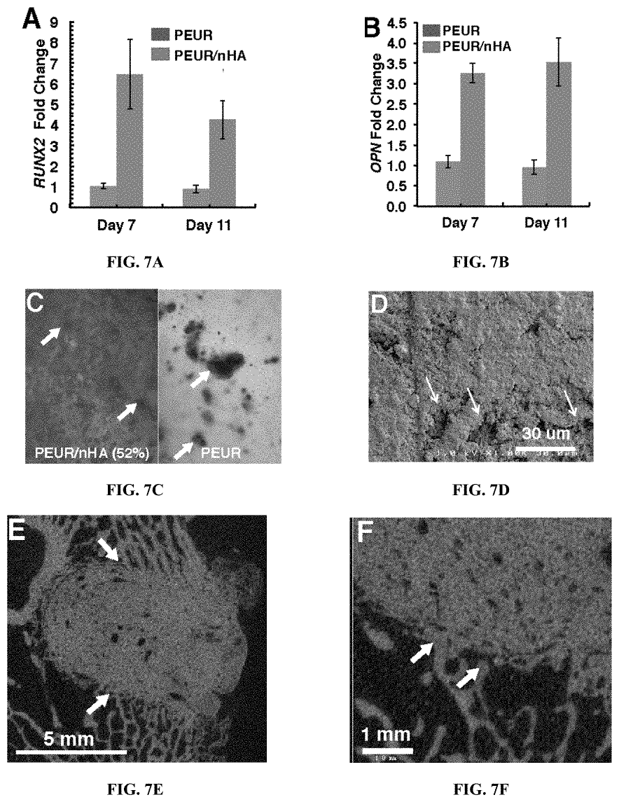

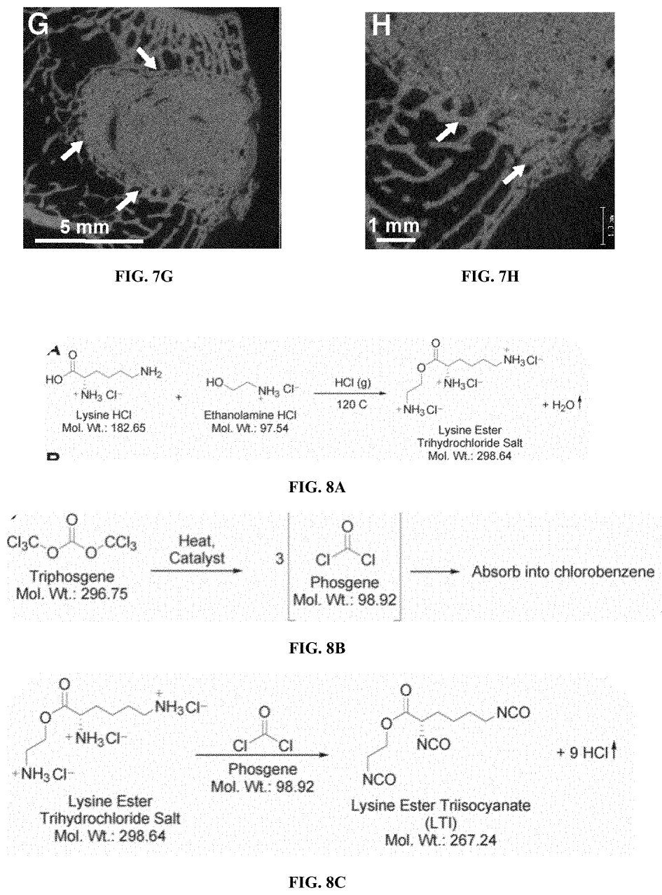

While nHA particles enhance osteogenic differentiation and new bone formation, the effects of nHA on remodeling of polymeric composites are less well known. This Example investigated the effects of nHA on osteogenic differentiation, mineralization, and in vivo remodeling in a rabbit femoral condyle plug defect model (FIG. 7A-B). In these experiments, nHA was added as a powder to the reactive PEUR mixture and was not reacted to form a nHA-LTI prepolymer in a separate step. Expression of the transcription factor Runx2, which stimulates osteoblast differentiation, and OPN, a late marker of osteoblast differentiation, were significantly increased on PEUR/nHA composites compared to PEUR (FIG. 7A-B). In a proof-of-concept experiment, PEUR/nHA composites were injected into 6×11 mm defects in the femoral condyles of rabbits. A representative μCT image shows remodeling of the composite (white arrows) near the host-bone interface at 6 weeks.

Similarly, at day 21 MC3T3 cells deposited more mineralized bone matrix on PEUR/nHA compared to PEUR (FIG. 7C). When co-cultured with rat bone marrow stromal cells for up to 21 days, RAW 264 monocytes differentiated to form osteoclasts that resorbed PEUR/nHA, as evidenced by the formation of resorption pits (white arrows in FIG. 7D). In contrast, differentiation of RAW 264 cells to multi-nucleated, TRAP-positive osteoclasts could not be induced on PEUR alone. PTKUR/nHA composites were injected into 6×11 mm defects in the femoral condyles of rabbits to investigate remodeling of these materials in vivo. Representative μCT images show remodeling of the composite (white arrows) near the host bone interface at 6 and 12 weeks (FIG. 7E-H). Similar to the PEUR/CG composites, appositional new bone formation and bone densification were observed near the interface. These data show that addition of nHA to polyurethanes enhances bone cell activity, and that polyurethane/nHA composites remodel in vivo.

### Example 12

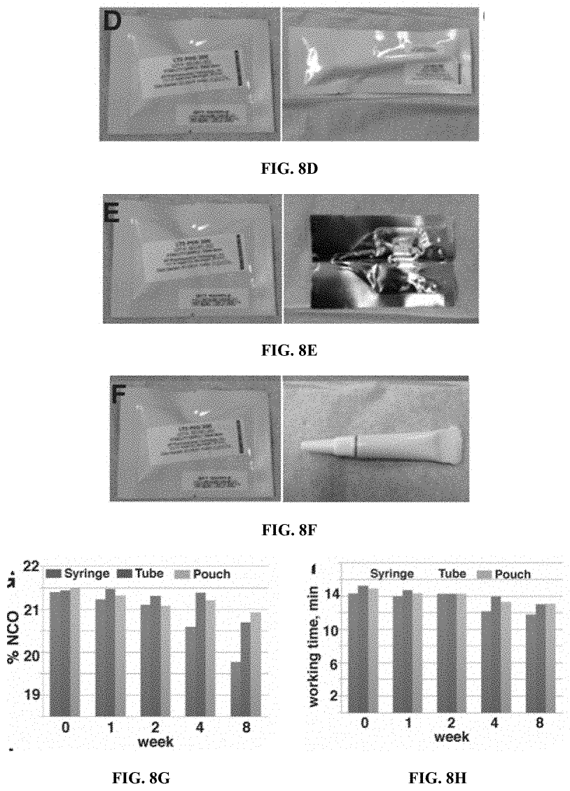

In this example, LTI is produced by a less hazardous, cost-effective, and environmentally friendly process based on catalytic decomposition of triphosgene to phosgene. Lysine ester trihydrochloride salt was synthesized from lysine hydrochloride and ethanolamine hydrochloride and recrystallized (FIG. 8A). A solution of phosgene in chlorobenzene was prepared by catalytic decomposition of triphosgene (FIG. 8B) and reacted with lysine ester trihydrochloride to prepare LTI (FIG. 8C). The triphosgene process resulted in high-purity (>97%) material that was purified by vacuum distillation to remove the chlorobenzene and carbon treatment to remove high-boiling oligomers and acids. This new process realized a five-fold reduction in raw material costs compared to the diphosgene process. PEG-LTI prepolymer was dispensed into three types of packaging: cyclic olefin polymer syringes (FIG. 8D), foil laminate pouches (FIG. 8E), and foil laminate tubes (FIG. 8F) and stored at 60° C. for up to 8 weeks. Foil laminate pouches and tubes provided better resistance to water compared to the syringes, which were permeable to water, resulting in a decrease in % NCO (FIG. 8G) and working time (FIG. 8H, measured by rheometry¹⁴).

### Example 13

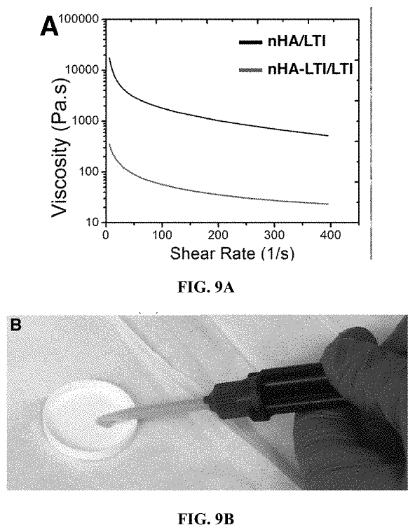

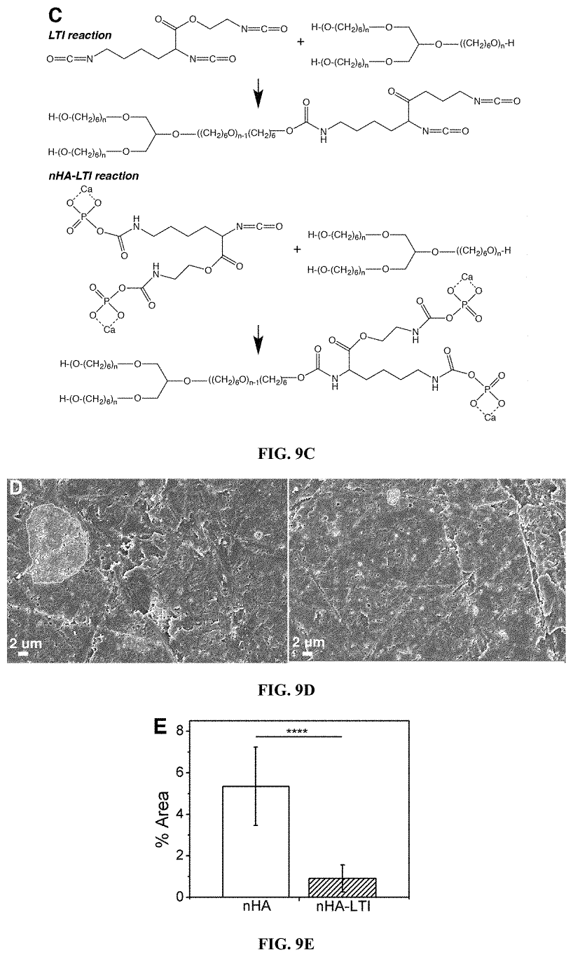

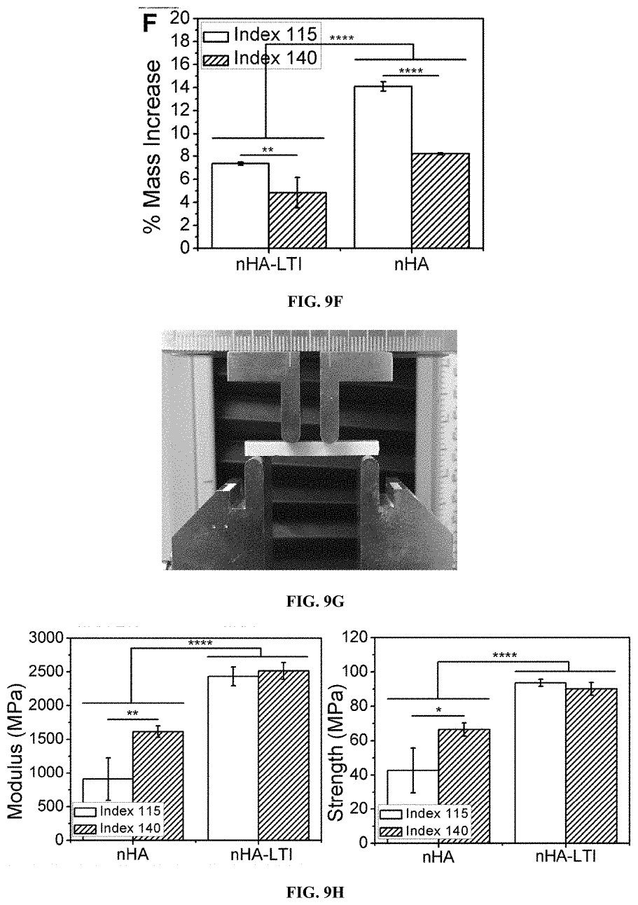

Both nHA and nHA-LTI nanoparticles were dispersed in LTI at 65 wt %. The resulting suspensions were shear-thinning, as evidenced by the decrease in viscosity with increasing shear rate (FIG. 9A). Furthermore, the viscosity of nHA-LTI/LTI was almost two orders of magnitude lower than that of nHA/LTI, which is consistent with the notion that grafting LTI to the nHA increases colloidal stability, resulting in a more homogeneous dispersion. At relevant shear rates (1-10 s⁻¹)(REF), the nHA-LTI/LTI prepolymer (65 wt % nHA) exhibited kinematic viscosity <20,000 cSt, which enabled it to be injected. To fabricate the bone cements, nHA/LTI or nHA-LTI/LTI prepolymer was mixed with poly(ε-caprolactone) triol (PCL triol, 300 g mol⁻¹) using a double-barrel syringe fitted with a static mixer (MedMix, FIG. 9B). The isocyanate index (ratio of NCO:OH equivalents*100) was either 115 or 140. The NCO groups in LTI and nHA-LTI react with hydroxyl groups in the PCL triol to form crosslinked organic-inorganic hybrid cements (FIG. 9C). Dispersion of nHA and nHA-LTI in the cements was evaluated by SEM (FIG. 9D). The area percentage of nHA-LTI aggregates was 5 times smaller than that measured for nHA (FIG. 9E), which is consistent with the rheology data (FIG. 9A) finding that nHA-LTI is more effectively dispersed in the cement. Swelling (assessed by incubating the cements in water for 24 h) decreased significantly with LTI grafting and increasing isocyanate index (FIG. 9F), which further suggests that surface grafting enhanced dispersion and crosslinking.

Four-point bending properties of nHA and nHA-LTI cements were measured according to ISO 5833, the international standard for PMMA, at Index 115 and 140 (FIG. 9G). The effects of the isocyanate index on bending strength and modulus were significant for nHA but not nHA-LTI cements. Surface grafting significantly increased cement bending modulus and bending strength 20-50% at both indices compared to no grafting (FIG. 9H). For quasi-static compression testing, cements were cured in 6 mm cylindrical tubes, cut to 12 mm, and soaked in PBS at 37° C. for 24 hours prior to testing. Yield strength of nHA-LTI cement increased with nHA-LTI loading up to 52 wt % (65 wt % nHA-LTI in nHA-LTI/LTI prepolymer) (FIG. 9I). Furthermore, nHA-LTI cement with >26 wt % nHA exhibited higher compressive strength than nHA cement with 52 wt % nHA. Similar trends were observed for Young's modulus. The mechanical properties of nHA-LTI cement exceeded the standard requirements for non-resorbable PMMA, including compressive strength of 70-90 MPa, compressive modulus of 2000-3000 MPa, and bending strength >80 MPa.

### Example 14

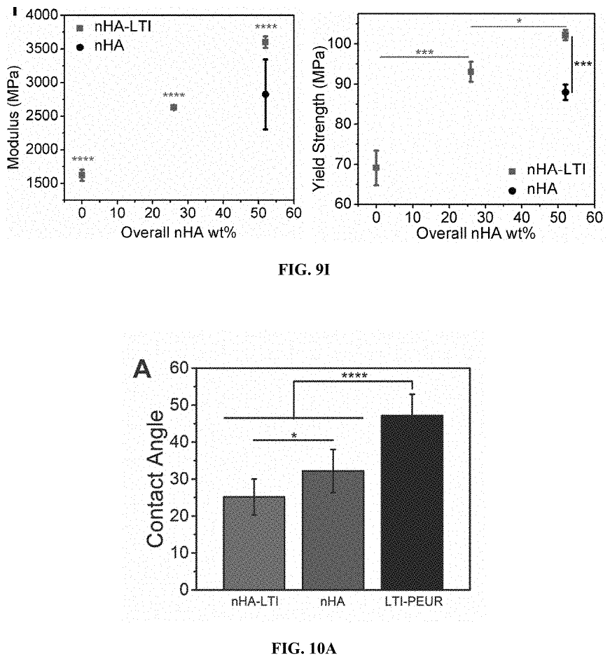

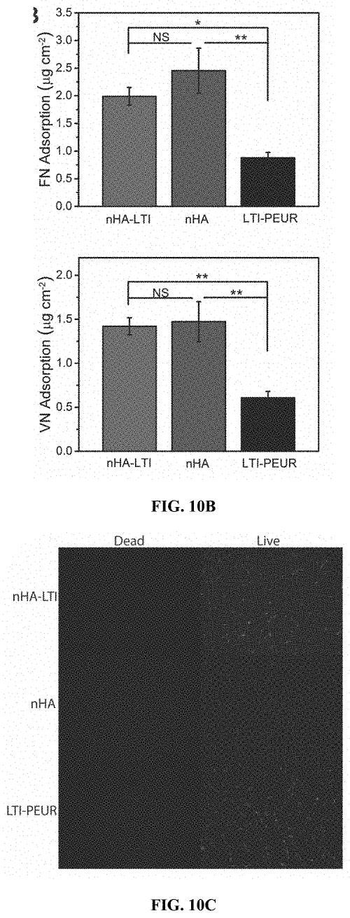

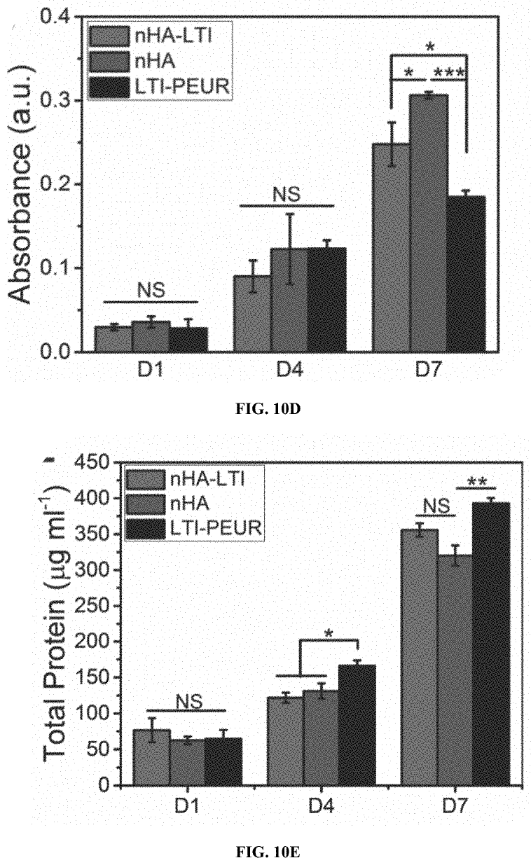

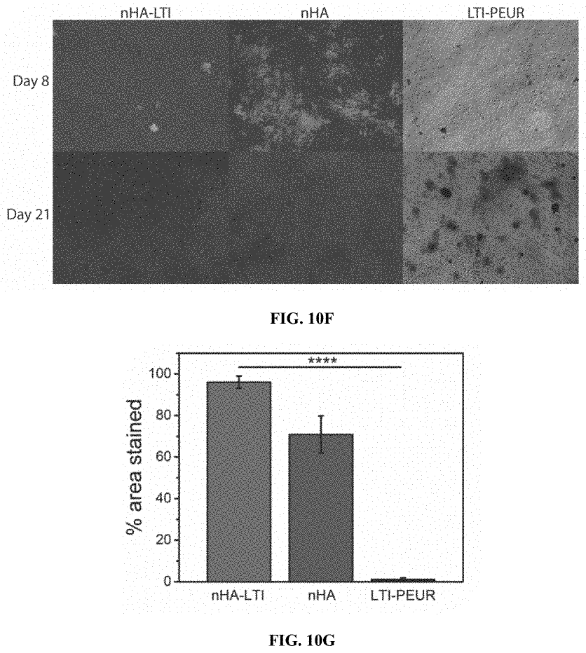

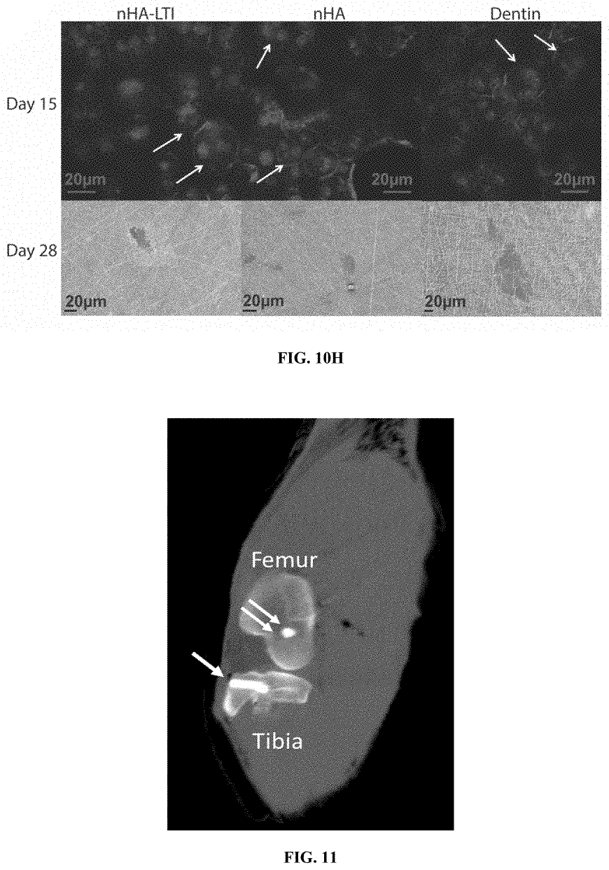

To evaluate the effect of nHA-LTI grafting on the biological properties of the cements, water contact angle (FIG. 10A) and protein adsorption of fibronectin and vitronectin (FIG. 10B) were measured. LTI-poly(ester urethane) (LTI-PEUR) without nHA had a contact angle of 50°, while the contact angle of hydroxyapatite is 10°. With 30 vol % (52 wt %) nHA loading, nHA-LTI cement (contact angle=25.2°±4.9°) was more hydrophilic than nHA cement (contact angle=32.2°±5.8°), suggesting that that the more homogenous dispersion of nHA-LTI rendered the surface more like HA. Specimens were also incubated in 5 μg/ml fibronectin or vitronectin solutions at 37° C. and protein adsorption measured using a Pierce BCA kit. The cements exhibited a two-fold increase in fibronectin and vitronectin adsorption compared to the LTI-PEUR control due to hydrophilicity. Fibronectin adsorption was comparable to that reported for pure HA with similar grain size, while vitronectin adsorption was lower than that reported for HA.

Cell viability, proliferation, and differentiation of mouse MC3T3 pre-osteoblasts on the cements was also assessed. MC3T3 cells were suspended in complete medium and seeded onto the substrates. Minimal (<5%) cell death was detected 48 h after cell seeding in all groups, indicating that the materials are non-toxic (FIG. 10C). Total protein increased from Day 1 to 7 for all groups, thereby indicating that cells proliferated on the surface (FIG. 10D). Proliferation was significantly higher on the LTI-PEUR control compared to the cements, but differences in proliferation between the nHA and nHA-LTI groups were insignificant. Cell proliferation assessed by the MTS assay showed significant differences between groups on day 7, with the highest cell numbers on the nHA cement (FIG. 10E).

To assess osteogenic differentiation and mineralization, MC3T3 cells were cultured in osteogenic medium (complete α-MEM supplemented with 10 nM dexamethasone, 50 μg ml⁻¹ ascorbic acid, and 0.1 mM glycerophosphate) for up to 21 days. RNA was extracted at 24 and 48 hours after induction to quantify gene expression of osteogenic differentiation markers using real-time PCR. Mineralization was assessed by Alizarin Red staining on days 8 and 21 (FIG. 10F). Staining was quantified by extraction of Alizarin Red from the substrates (FIG. 10G) and by measuring the area % of stained surface (FIG. 10H). On day 8, nHA-LTI showed both increased absorption and area % stained compared to nHA, while the LTI-PEUR control showed minimal staining. On day 21, nodules of mineralized matrix were observed on LTI-PEUR, while the entire surface of both nHA and nHA-LTI cements stained positive for Alizarin Red. To evaluate osteoclast-mediated resorption of the cements, MC3T3 cells were co-cultured with RAW 264.7 cells in osteogenic medium supplemented with 10 nM Vitamin D 3 to stimulate RAW 264.7 cells to differentiate to osteoclasts. Actin (red)/DAPI (blue nucleus) staining was performed on day 15. Osteoclasts were identified as multi-nucleated cells with an actin ring (FIG. 10I). Resorption pits on the surface of nHA and nHA-LTI cements as well as the dentin control were detected by SEM on day 28 (FIG. 10J). The osteoclasts formed on the dentin positive surface appeared to be larger with more nuclei than osteoclasts detected on the cements, and resorption pits on nHA and nHA-LTI cements were smaller than those on dentin. No evidence of osteoclasts or resorption was observed on the surface of LTI-PEUR.

The mineralization and resorption behavior of cells cultured on the cements were dramatically different than that on polymer, indicating that the cements behaved more like ceramics. In view thereof, the maximum amount of ceramic component in the cements was pushed to 52 wt % for maximum biological performance, and still achieved high mechanical performance comparable to that of biologically inert PMMA.

In addition to being biologically inert, the current PMMA bone cements have other issues, for example, the preparation of PMMA cements involves mixing a liquid phase with powder and waiting for the viscosity of the system to become workable. Once the polymerization process of PMMA is initialized by mixing, the workable time is very limited. Failure to work PMMA cements within the working time results in leakage of toxic monomer to the surrounding tissue or filling the defect inadequately. The instant nHA-LTI cement solves this handling problem of PMMA cement, since the nHA-LTI cement is based on two liquid phases with moderate viscosity that can be combined and mixed spontaneously when injected through a static mixer. Setting time can be tailored by adjusting the amount of FeAA catalyst. If necessary, nHA-LTI particles can be washed off from the prepolymer by organic solvents and re-dispersed into LTI, forming catalyst-free dispersion. The catalyst needed for the crosslinking reaction can be incorporated into PCL side.

In this study, injectable and resorbable nanocrystalline hydroxyapatite-PEUR hybrid cements exhibiting bone-like strength comparable to the current ISO standard of PMMA bone cements were developed. The material was synthesized by first grafting LTI onto nHA to form a nHA-LTI/LTI prepolymer and further crosslinking with PCL 300 to yield nHA-LTI hybrid cement. The bonding between surface OH group of nHA and N═C=O group of LTI was characterized by FTIR and XPS, and enhanced dispersion of nHA in the polymer matrix. Neither the crystallinity nor grain size of nHA was changed by grafting. Homogenous dispersion of nHA in nHA-LTI cement contributed to bone-like strength and enhanced protein adsorption and osteogenic differentiation of MC3T3 cells. The cement also supported osteoclastogenic differentiation and was resorbable by osteoclasts. nHA-PEUR cements provide favorable handling properties (injectability and settability), exhibit strengths exceeding those of trabecular bone and weight-bearing PMMA, enhance osteoblast differentiation and mineralization, and support osteoclast-mediated bone resorption. These findings support the potential of nHA/PEUR hybrid cements for repair and restoration of bone defects at weight-bearing sites.

### Example 15

Experimental Design: nHA-LTI/PCL300t composites with MG or MG/BG blended matrix particles were tested in the weight-bearing sheep model for their ability to withstand mechanical loading in the cellularly active wound healing environment. nHA-LTI prepolymer was prepared by mixing 65 wt % nHA nanoparticles with LTI in a speed mixer for 1 minute. Then, 0.03 wt % FeAA catalyst (in ε-caprolactone) was added to the mixing cup and mixed for 9 minutes for a total of 10 minutes speed mixing. The prepolymer continued to react at 50° C. for 3 hours.

The materials were tested in a weight-bearing tibial plateau slot defect and a non-weight bearing femoral plug defect in the hind limbs of 8 skeletally mature sheep (for n=8/formulation). Two 8 mm cylindrical defects, 16 mm deep were created in the medial and lateral condyles of each hind limb using a surgical drill. To create the tibial defects, the tibia was exposed taking care to preserve surrounding soft tissue. A slot defect across the entire width of the anterior portion of the tibia, 6 mm in height, and approximately 50% of the total anterior to posterior tibial depth was created leaving a thin shelf above the defect area (FIG. 11). Composites were fabricated in the surgical suite as discussed previously. MG or a blend of MG/BG particles were mixed with a PCL and catalyst phase. Then, the nHA-LTI prepolymer was added and the material molded into the defects by hand. The wound was closed and composites cured within 10 minutes of adding the prepolymer. Animals were kept in a sling for 72 hours postoperatively to prevent early weight-bearing.

Animals were sacrificed at 16 weeks unless CT analysis indicated fracture or discomfort was apparent.

Preliminary Results: In vitro data suggested enhanced bioactivity and bone-like strength with the incorporation of nHA in the microstructure of a nHA-LTI/PCL PUR.

Four animals tolerated the experimental grafts up to the 16 week time point. Both CT and μCT showed maintenance of the defect spaces with MG formulations and μCT images indicated trabecular infiltration of these grafts. Fragmentation of the MG/BG formulation in the tibial defect was evident in CT and μCT images; however, grafts appeared stable in non-weight-bearing femoral condyle defects. Histology sections revealed high cellular activity in tibial defects with MG/BG and grafts were almost completely replaced with fibrous tissue or voids. This difference amongst defect sites validates the weight-bearing defect is much more cellularly active. PUR/MG groups were well tolerated in the plateau defect. A creeping substitution mechanism was evident at the periphery of the grafts where the material was well integrated with the host bone although infiltration and remodeling was slower than anticipated.

These preliminary results suggest that a degradable PUR/ceramic composite has potential for use in a weight-bearing site.

Throughout the specification, various publications are referenced. All such references, including those listed below, are incorporated herein by reference.

### REFERENCES

1 Kruger, R., Seitz, J. M., Ewald, A., Bach, F. W. & Groll, J. Strong and tough magnesium wire reinforced phosphate cement composites for load-bearing bone replacement. J Mech Behav Biomed Mater 20, 36-44, (2013).
2 Kruger, R. & Groll, J. Fiber reinforced calcium phosphate cements—on the way to degradable load bearing bone substitutes? Biomaterials 33, 5887-5900, (2012).
3 Dumas, J. E., Prieto, E. M., Zienkiewicz, K. J., Guda, T., Wenke, J. C., Bible, J., Holt, G. E. & Guelcher, S. A. Balancing the rates of new bone formation and polymer degradation enhances healing of weight-bearing allograft/polyurethane composites in rabbit femoral defects. Tissue Eng Part A 20, 115-129, (2014).
4 Dumas, J. E., Davis, T. E., Yoshii, T., Nyman, J., Holt, G. E., Perrien, D. S., Boyce, T. M. & Guelcher, S. A. Synthesis of Allograft Bone/Polymer Composites and Evaluation of Remodeling in a Rabbit Femoral Condyle Model. Acta Biomaterialia 6, 2394-2406, (2010).
5 Prieto, E. M., Talley, A. D., Gould, N. R., Zienkiewicz, K. J., Drapeau, S. J., Kalpakci, K. N. & Guelcher, S. A. Effects of particle size and porosity on in vivo remodeling of settable allograft bone/polymer composites. J Biomed Mater Res B Appl Biomater, (2015).
6 Harmata, A. J., Uppuganti, S., Granke, M., Guelcher, S. A. & Nyman, J. S. Compressive fatigue and fracture toughness behavior of injectable, settable bone cements. J Mech Behav Biomed Mater 51, 345-355, (2015).
6 Harmata, A. J., Ward, C. L., Zienkiewicz, K., Wenke, J. C. & Guelcher, S. A. Investigating the Effects of Surface-Initiated Polymerization of ε-Caprolactone to Bioactive Glass Particles on the Mechanical Properties of Settable Polymer/Ceramic Composites. J Mater Res 2014, 20-30, (2014).
8 Chan, C., Thompson, I., Robinson, P., Wilson, J. & Hench, L. Evaluation of Bioglass/dextran composite as a bone graft substitute. Int J Oral Maxillofac Surg 31, 73-77, (2002).
9 Lorden, E. R., Miller, K. J., Bashirov, L., Ibrahim, M. M., Hammett, E., Jung, Y., Medina, M. A., Rastegarpour, A., Selim, M. A., Leong, K. W. & Levinson, H. Mitigation of hypertrophic scar contraction via an elastomeric biodegradable scaffold. Biomaterials 43, 61-70, (2015).
10 Dumas, J. E., Zienkiewicz, K., Tanner, S. A., Prieto, E. M., Bhattacharyya, S. & Guelcher, S. Synthesis and Characterization of an Injectable Allograft Bone/polymer Composite Bone Void Filler with Tunable Mechanical Properties. Tissue Eng Part A 16, 2505-2518, (2010).
11 Dumas, J. E., Brownbaer, P. B., Prieto, E. M., Guda, T., Hale, R. G., Wenke, J. C. & Guelcher, S. A. Injectable reactive biocomposites for bone healing in critical-size rabbit calvarial defects. Biomed Mater 7, 024112, (2012).
12 Martin, J. R., Gupta, M. K., Page, J. M., Yu, F., Davidson, J. M., Guelcher, S. A. & Duvall, C. L. A porous tissue engineering scaffold selectively degraded by cell-generated reactive oxygen species. Biomaterials 35, 3766-3776, (2014).
13 Laurencin, C. T., Kumbar, S. G. & Nukavarapu, S. P. Nanotechnology and orthopedics: a personal perspective. Wiley Interdiscip Rev Nanomed Nanobiotechnol 1, 6-10, (2009).
14 Sun, F., Zhou, H. & Lee, J. Various preparation methods of highly porous hydroxyapatite/polymer nanoscale biocomposites for bone regeneration. Acta Biomater 7, 3813-3828, (2011).
15 Ngiam, M., Liao, S., Patil, A. J., Cheng, Z., Chan, C. K. & Ramakrishna, S. The fabrication of nano-hydroxyapatite on PLGA and PLGA/collagen nanofibrous composite scaffolds and their effects in osteoblastic behavior for bone tissue engineering. Bone 45, 4-16, (2009).
16 Webster, T. J., Ergun, C., Doremus, R. H., Siegel, R. W. & Bizios, R. Enhanced osteoclast-like cell functions on nanophase ceramics. Biomaterials 22, 1327-1333, (2001).
17 Webster, T. J., Ergun, C., Doremus, R. H., Siegel, R. W. & Bizios, R. Enhanced functions of osteoblasts on nanophase ceramics. Biomaterials 21, 1803-1810, (2000).
18 Eckert, H. & Auerweck, J. Solvent-Free and Safe Process for the Quantitative Production of Phosgene from Triphosgene by Deactivated Imino-Based Catalysts. Organic Process Research & Development 14, 1501-1505, (2010).
19 LeGeros, R. Properties of osteoconductive biomaerials: calcium phosphates. Clin Orthop Relat Res 395, 81-98, (2002).
20 Yoshii, T., Dumas, J. E., Okawa, A., Spengler, D. M. & Guelcher, S. A. Synthesis, characterization of calcium phosphates/polyurethane composites for weight-bearing implants. J Biomed Mater Res B Appl Biomater 100, 32-40, (2012).
21 Page, J. M., Prieto, E. M., Dumas, J. E., Zienkiewicz, K. J., Wenke, J. C., Brown-Baer, P. & Guelcher, S. A. Biocompatibility and chemical reaction kinetics of injectable, settable polyurethane/allograft bone biocomposites. Acta Biomater 8, 4405-4416, (2012).
22 Kuehn, K.-D. Bone Cements. (Springer, 2000).
23 MacMillan, A. K., Lamberti, F. V., Moulton, J. N., Geilich, B. M. & Webster, T. J. Similar healthy osteoclast and osteoblast activity on nanocrystalline hydroxyapatite and nanoparticles of tri-calcium phosphate compared to natural bone. International journal of nanomedicine 9, 5627-5637, (2014).
24 Liu, Q., de Wijn, J. R., De Groot, K. & van Blitterswijk, C. C. Surface modification of nano-apatite by grafting organic polymer. Biomaterials 19, 1067-1072, (1998).
25 Kickelbick, G. Concepts for the incorporation of inorganic building blocks into organic polymers on a nanoscale. Progress in Polymer Science 28, 83-114, (2003).
26 Hafeman, A. E., Zienkiewicz, K. J., Zachman, A. L., Sung, H. J., Nanney, L. B., Davidson, J. M. & Guelcher, S. A. Characterization of the degradation mechanisms of lysine-derived aliphatic poly(ester urethane) scaffolds. Biomaterials 32, 419-429, (2011).
27 Harmata, A. J., Ma, Y., Sanchez, C. J., Ziennkiewicz, K. J., Elefteriou, F., Wenke, J. C. & Guelcher, S. A. Effects of D-amino Acid Biofilm Dispersal Agents on In Vitro Osteocompatibility and New Bone Formation in an Ovine Model. Clin Orthop Relat Res In Press, (2015).
28 Harmata, A. J., Uppuganti, S., Granke, M., Guelcher, S. A. & Nyman, J. S. Compressive fatigue and fracture toughness behavior of injectable, settable bone cements. (Submitted).
29 Brown, K. V., Li, B., Guda, T., Perrien, D. S., Guelcher, S. A. & Wenke, J. C. Improving bone formation in a rat femur segmental defect by controlling bone morphogenetic protein-2 release. Tissue engineering. Part A 17, 1735-1746, (2011).
30 Guo, R., Lu, S., Page, J. M., Merkel, A. R., Basu, S., Sterling, J. A. & Guelcher, S. A. Fabrication of 3D Scaffolds with Precisely Controlled Substrate Modulus and Pore Size by Templated-Fused Deposition Modeling to Direct Osteogenic Differentiation. Advanced healthcare materials In Press, (2015).
31 Guo, R., Merkel, A., Sterling, J. A., Davidson, J. M. & Guelcher, S. A. Substrate Modulus of 3D-Printed Scaffolds Regulates the Regenerative Response in Subcutaneous Implants through the Macrophage Phenotype and Wnt Signaling. Submitted.
32 Guo, R., Ward, C. L., Davidson, J. M., Duvall, C. L., Wenke, J. C. & Guelcher, S. A. A transient cell-shielding method for viable MSC delivery within hydrophobic scaffolds polymerized in situ. Biomaterials 54, 21-33, (2015).
33 Goto, K., Shinzato, S., Fujibayashi, S., Tamura, J., Kawanabe, K., Hasegawa, S., Kowalski, R. & Nakamura, T. The biocompatibility and osteoconductivity of a cement containing beta-TCP for use in vertebroplasty. J Biomed Mater Res A 78, 629-637, (2006).
34 Kokubo, T. & Takadama, H. How useful is SBF in predicting in vivo bone bioactivity? Biomaterials 27, 2907-2915, (2006).
35 Zhao, Q. H., McNally, A. K., Rubin, K. R., Renier, M., Wu, Y., Rose-Caprara, V., Anderson, J. M., Hiltner, A., Urbanski, P. & Stokes, K. Human plasma alpha2-macroglobulin promotes in vitro oxidative stress cracking of Pellethane 2363-80A: In vivo and in vitro correlations. J Biomed Mater Res 27, 379-389, (1993).
Rathbone, C. R., Cross, J. D., Brown, K. V., Murray, C. K. & Wenke, J. C. Effect of various concentrations of antibiotics on osteogenic cell viability and activity. J Orthop Res 29, 1070-1074, (2011).
37 Elefteriou, F., Benson, M. D., Sowa, H., Starbuck, M., Liu, X., Ron, D., Parada, L. F. & Karsenty, G. ATF4 mediation of NF1 functions in osteoblast reveals a nutritional basis for congenital skeletal dysplasiae. Cell metabolism 4, 441-451, (2006).
38 Elefteriou, F., Ahn, J. D., Takeda, S., Starbuck, M., Yang, X., Liu, X., Kondo, H., Richards, W. G., Bannon, T. W., Noda, M., Clement, K., Vaisse, C. & Karsenty, G. Leptin regulation of bone resorption by the sympathetic nervous system and CART. Nature 434, 514-520, (2005).
39 Takahashi, N., Akatsu, T., Udagawa, N., Sasaki, T., Yamaguchi, A., Moseley, J. M., Martin, T. J. & Suda, T. Osteoblastic cells are involved in osteoclast formation. Endocrinology 123, 2600-2602, (1988).
40 Yao, Z., Xing, L., Qin, C., Schwarz, E. M. & Boyce, B. F. Osteoclast precursor interaction with bone matrix induces osteoclast formation directly by an interleukin-1-mediated autocrine mechanism. J Biol Chem 283, 9917-9924, (2008).
41 Jensen, E. D., Pham, L., Billington, C. J., Jr., Espe, K., Carlson, A. E., Westendorf, J. J., Petryk, A., Gopalakrishnan, R. & Mansky, K. Bone morphogenic protein 2 directly enhances differentiation of murine osteoclast precursors. J Cell Biochem 109, 672-682, (2010).
42 Domaschke, H., Gelinsky, M., Burmeister, B., Fleig, R., Hanke, T., Reinstorf, A., Pompe, W. & Rosen-Wolff, A. In vitro ossification and remodeling of mineralized collagen I scaffolds. Tissue Eng 12, 949-958, (2006).
43 Schilling, A. F., Linhart, W., Filke, S., Gebauer, M., Schinke, T., Rueger, J. M. & Amling, M. Resorbability of bone substitute biomaterials by human osteoclasts. Biomaterials 25, 3963-3972, (2004).
44 Karp, J. M., Rzeszutek, K., Shoichet, M. S. & Davies, J. E. Fabrication of precise cylindrical three-dimensional tissue engineering scaffolds for in vitro and in vivo bone engineering applications. J Craniofac Surg 14, 317-323, (2003).
45 Li, B., Yoshii, T., Hafeman, A. E., Nyman, J. S., Wenke, J. C. & Guelcher, S. A. The effects of rhBMP-2 released from biodegradable polyurethane/microsphere composite scaffolds on new bone formation in rat femora. Biomaterials 30, 6768-6779, (2009).
46 Brown, K. V., Li, B., Guda, T., Perrien, D. S., Guelcher, S. A. & Wenke, J. C. Improving Bone Formation in a Rat Femur Segmental Defect by Controlling BMP-2 Release. Tissue Eng Part A, (In Press).
47 Bouxsein, M. L., Boyd, S. K., Christiansen, B. A., Guldberg, R. E., Jepsen, K. J. & Muller, R. Guidelines for assessment of bone microstructure in rodents using micro-computed tomography. J Bone Miner Res 25, 1468-1486, (2010).
48 Page, J. M., Prieto, E. M., Dumas, J. E., Zienkiewicz, K. J., Wenke, J. C., BrownBaer, P. & Guelcher, S. A. Reactivity and Biocompatibility of Injectable Polyurethane/Allograft Bone Biocomposites. (Submitted).
49 Dumas, J. E., Prieto, E. M., Guda, T., Zienkiewicz, K. J., Garza, J., Bible, J., Holt, G. E. & Guelcher, S. A. Remodeling of Settable Weight-bearing Allograft Bone/Polymer Composites Carrying Recombinant Human Bone Morphogenetic Protein-2 (rhBMP-2) in Rabbit Femora. (Submitted).
50 S. Ramakrishna, J. Mayer, E. Wintermantel, K. W. Leong, Composites Science and Technology 2001, 61, 1189.
51 J. A. Hall, M. J. Beuerlein, M. D. McKee, J Bone Joint Surg Am 2009, 91 Suppl 2 Pt 1, 74; V. Musahl, I. Tarkin, P. Kobbe, C. Tzioupis, P. A. Siska, H.-C. Pape, The Journal of Bone & Joint Surgery 2009, 91B 426; Y. MJ, B. R L, Orthop Rev 1994, 23, 149; S. J, M. R, B. D, Clin Orthop 1976, 138, 94; P. PJ, P. AA, T. GS, M. AF, K. DS, S. PN, Injury 2006, 37, 475.
52 K.-D. Kuehn, Bone Cements, Springer, Berlin 2000.
53 A. J. W. Johnson, B. A. Herschler, Acta Biomaterialia 2011, 7, 16.
54 R. Kruger, J. Groll, Biomaterials 2012, 33, 5887.
55 J. E. Dumas, E. M. Prieto, K. J. Zienkiewicz, T. Guda, J. C. Wenke, J. Bible, G. E. Holt, S. A. Guelcher, Tissue engineering. Part A 2014, 20, 115; S. Gay, S. Arostegui, J. Lemaitre, Materials Science & Engineering C-Biomimetic and Supramolecular Systems 2009, 29, 172.
56 J. E. Dumas, T. E. Davis, T. Yoshii, J. Nyman, G. E. Holt, D. S. Perrien, T. M. Boyce, S. A. Guelcher Acta Biomaterialia 2010, 6, 2394.
57 R. Kruger, J. M. Seitz, A. Ewald, F. W. Bach, J. Groll, J Mech Behav Biomed Mater 2013, 20, 36.
58 G. Kickelbick, Progress in Polymer Science 2003, 28, 83.
59 M. Supova, J Mater Sci Mater Med 2009, 20, 1201.
60 L. Y. Jiang, C. D. Xiong, D. L. Chen, L. X. Jiang, X. B. Pang, Applied Surface Science 2012, 259, 72; L. X. Jiang, L. Y. Jiang, L. J. Xu, C. T. Han, C. D. Xiong, Express Polymer Letters 2014, 8, 133; X. Y. Qiu, L. Chen, J. L. Hu, J. R. Sun, Z. K. Hong, A. X. Liu, X. S. Chen, X. B. Jing, Journal of Polymer Science Part a-Polymer Chemistry 2005, 43, 5177.
61 L. Y. Jiang, L. X. Jiang, C. D. Xiong, L. J. Xu, Y. Li, Journal of Biomaterials Applications 2016, 30, 750.
62 H. J. Lee, S. E. Kim, H. W. Choi, C. W. Kim, K. J. Kim, S. C. Lee, European Polymer Journal 2007, 43, 1602.
53 S. J. Dong, T. Yu, J. C. Wei, X. B. Jing, Y. M. Zhou, P. B. Zhang, X. S. Chen, Chemical Journal of Chinese Universities-Chinese 2009, 30, 1018; R. R. Fan, L. X. Zhou, W. Song, D. X. Li, D. M. Zhang, R. Ye, Y. Zheng, G. Guo, International Journal of Biological Macromolecules 2013, 59, 227.
64 Z. L. Wang, Y. Xu, Y. Wang, Y. Ito, P. B. Zhang, X. S. Chen, Biomacromolecules 2016, 17, 818.
65 H. Tanaka, T. Watanabe, M. Chikazawa, K. Kandori, T. Ishikawa, J Coll Interfac Sci 1998, 206, 205; Q. Liu, J. R. de Wijn, K. De Groot, C. C. van Blitterswijk, Biomaterials 1998, 19, 1067; G.-C. Dong, J.-S. Sun, C.-H. Yao, G. J. Jiang, C.-W. Huang, F.-H. Lin, Biomaterials 2001, 22, 3179.
66 G. C. Dong, J. S. Sun, C. H. Yao, G. J. Jiang, C. W. Huang, F. H. Lin, Biomaterials 2001, 22, 3179.
67 D. Aronov, R. Rosen, E. Z. Ron, G. Rosenman, Process Biochemistry 2006, 41, 2367.
68 T. J. Webster, C. Ergun, R. H. Doremus, R. W. Siegel, R. Bizios, Journal of Biomedical Materials Research 2000, 51, 475.
69 R. Vaishya, M. Chauhan, A. Vaish, Journal of Clinical Orthopaedics and Trauma 2013, 4, 157.

What is claimed is:

1. A hybrid composite, comprising:
nanocrystalline hydroxyapatite (nHA);
lysine-derived polyisocyanate;
a thioketal, polyester triol, or poly(ε-caprolactone) triol; and
a porogen at a concentration of between 0 and 50 wt %;
wherein the nHA is grafted to the lysine-derived polyisocyanate; and
wherein the composite exhibits less than 3% nHA aggregates.

2. The composite of claim 1, wherein the composite is at least one of resorbable, injectable, settable, and moldable.

3. The composite of claim 1, wherein the composite includes at least one additive.

4. The composite of claim 3, wherein the at least one additive is ceramic granules.

5. The composite of claim 4, wherein the ceramic granules comprise slowly degrading ceramic granules having a size of between 100 and 300 μm.

6. The composite of claim 5, wherein the ceramic granules are arranged and disposed to facilitate osseointegration in a subject.

7. The composite of claim 1, comprising between 20 and 65 wt % nHA.

8. The composite of claim 1, further comprising at least one anti-microbial, at least one osteobiologic, or a combination thereof.

9. The composite of claim 1, wherein the composite is selected from the group consisting of a bone void filler, hydrolytically stable, oxidatively degradable, and combinations thereof.

10. The composite of claim 1, wherein the composite exhibits a contact angle of less than 30°.

11. The composite of claim 1, wherein the composite comprises a nitrogen:phosphorus ratio on the surface of the nHA of at least 0.6.

12. The composite of claim 1, wherein at least 40% of the hydroxyl groups on the nHA are grafted to the lysine-derived polyisocyanate.

13. A method for producing a polymer network, comprising:
reacting nanocrystalline hydroxyapatite (nHA) particles with lysine derived polyisocyanate and a thioketal (TK), polyester triol, or poly(ε-caprolactone) triol to form the polymer network;
wherein the nHA is grafted to the lysine-derived polyisocyanate during the reacting step; and
wherein the polymer network exhibits less than 3% nHA aggregates.

14. The method of claim 13, wherein the nHA particles include a size selected from the group consisting of <100 nm, a specific surface of greater than 10 m² g⁻¹, and a combination thereof.

15. The method of claim 13, wherein the nHA particles are reacted with the polyisocyanate at a NCO:OH ratio of between about 20:1 to about 3:1.

16. The method of claim 13, wherein the method further comprises the step of reacting the nHA with lysine derived triisocyanates (LTI) to form nHA-LTI prepolymers.

17. The method of claim 13, wherein the polymer network is 55% nHA.

18. The method of claim 13, wherein the TK diol is hydrolytically stable and oxidatively degradable.

19. The method of claim 13, wherein the TK diol includes thioketal bonds that are destabilized by hydroxyl radicals.

20. A method for producing a polymer network, comprising:
reacting nanocrystalline hydroxyapatite (nHA) particles with lysine derived triisocyanates (LTI) to form prepolymers; and
reacting the prepolymers with thioketal (TK), polyester triol, or poly(ε-caprolactone) triol to form the polymer network;
wherein the nHA is grafted to the lysine-derived polyisocyanate during the reacting step; and
wherein the prepolymer includes a viscosity of less than 1000 Pa*s.
